(19) United States
(12) Patent Application Publication
(10) Pub. No.: US 2004/0254185 A1
(43) Pub. Date: Dec. 16, 2004

(54) Selective anthranilamide pyridine amides as inhibitors of vegfr-2 and vegfr-3

(76) Inventors: Alexander Ernst, Berlin (DE); Andreas Huth, Berlin (DE); Martin Kruger, Berlin (DE); Karl-Heinz Thierauch, Berlin (DE); Andreas Menrad, Oranienburg (DE); Martin Haberey, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/477,119

(22) PCT Filed: May 3, 2002
(86) PCT No.: PCT/EP02/04924

(30) Foreign Application Priority Data
May 8, 2001 (DE) 101 23 574.7
May 15, 2001 (DE) 101 25 294.3
Dec. 21, 2001 (DE) 101 64 590.2

(51) Int. Cl. C 07D 4 1/02; C 07D 4 3/02; A61K 31/675

(52) U.S. Cl. 514/242; 514/252.03; 514/255.05; 514/256; 514/332; 544/182; 544/238; 544/329; 544/405; 546/256; 514/89

(57) ABSTRACT

Selective anthranilamide pyridinamides as VEGFR-2 and VEGFR-3 inhibitors, their production and use as pharmaceutical agents for treating diseases that are triggered by persistent angiogenesis are described. The compounds according to the invention can be used as or in the case of psoriasis, Kaposi's sarcoma, restenosis, such as, e.g., stent-induced restenosis, endometriosis, Crohn's disease, Hodgkin's disease, leukemia; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, and inhibition of the reocclusion of vessels after balloon catheter treatment, in vascular prosthetics or after mechanical devices are used to keep vessels open, such as, e.g., stents, as immunosuppressive agents, as a support in scar-free healing, senile keratosis and contact dermatitis. The compounds according to the invention can also be used as VEGFR-3 inhibitors in the case of lymphangiogenesis in hyper- and dysplastic changes of the lymphatic system.

The invention relates to selective anthranilamide pyridinamides as VEGFR-2 and VEGFR-3 inhibitors, their production and use as pharmaceutical agents for treating diseases that are triggered by persistent angiogenesis.

Persistent angiogenesis can be the cause of various diseases, such as psoriasis; arthritis, such as rheumatoid arthritis, hemangioma, endometriosis, angiofibroma; eye diseases, such as diabetic retinopathy, neovascular glaucoma; renal diseases, such as glomerulonephritis, diabetic nephropathy, malignant nephrosclerosis, thrombotic microangiopathic syndrome, transplant rejections and glomerulopathy; fibrotic diseases, such as cirrhosis of the liver, mesangial cell proliferative diseases and arteriosclerosis or can result in an aggravation of these diseases.

Persistent angiogenesis is induced by the factor VEGF via its receptor. So that VEGF can exert this action, it is necessary that VEGF bind to the receptor, and a tyrosine phosphorylation is induced.

Direct or indirect inhibition of the VEGF receptor (VEGF=vascular endothelial growth factor) can be used for treating such diseases and other VEGF-induced pathological angiogenesis and vascular permeable conditions, such as tumor vascularization. For example, it is known that the growth of tumors can be inhibited by soluble receptors and antibodies against VEGF.

Anthranilic acid amides that are used as pharmaceutical agents for treating psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, and for inhibiting the reocclusion of vessels after balloon catheter treatment, in vascular prosthetics or after mechanical devices are used to keep vessels open, such as, e.g., stents, are known from WO 00/27819.

Strong angiogenesis is a prerequisite for the proliferation of the extrauterine endometrium in the case of endometriosis. Angiogenesis inhibition can therefore also be used for the treatment of this form of disease that causes painful conditions and often results in infertility.

The known compounds are generally effective in the indications cited, but their effectiveness generally accompanies toxicity and an inferior compatibility of the medication.

There is therefore a desire, on the one hand, for more effective compounds, and, on the other hand, for more toxicologically harmless compounds, which, moreover, should also be more compatible.

It has now been found that compounds of general formula I

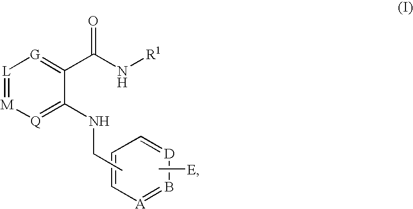

in which

A, B and D, independently of one another, stand for a nitrogen or carbon atom, whereby at least one nitrogen atom is contained in the ring,

E stands for aryl or hetaryl that is optionally substituted in one or more places in the same way or differently with halogen, cyano, C₁-C₆-alkyl, C₁-C₆-alkoxy, halo-C₁-C₆-alkyl or with the group —OR⁵, —SR⁴, —SOR⁴ or —SO₂R⁴, or for the group —COOR⁸, —CONR²R³, —SR⁴, —SOR⁴, —SO₂R⁴, —SCN, —PO(OR¹²)(OR¹³), —CH═CH—COR⁹ or —C≡C—R⁹,

G stands for a nitrogen atom or for the group —C—X,

L stands for a nitrogen atom or for the group —C—X,

M stands for a nitrogen atom or for the group —C—X,

Q stands for a nitrogen atom or for the group —C—X, whereby at most one nitrogen atom is in the ring,

X stands for hydrogen, halogen or for C₁-C₆-alkyl, C₁-C₆-alkyloxy or C₁-C₆-carboxyalkyl that is unsubstituted or optionally substituted in one or more places with halogen,

R¹ stands for branched or unbranched C₁-C₁₂-alkyl or C₂-C₁₂-alkenyl that is optionally substituted in one or more places in the same way or differently with halogen, hydroxy, C₁-C₆-alkyloxy, aralkyloxy, C₁-C₆-alkyl and/or with the group —NR²R³; or for C₃-C₁₀-cycloalkyl or C₃-C₁₀-cycloalkenyl that is optionally substituted in one or more places in the same way or differently with halogen, hydroxy, C₁-C₆-alkyloxy, C₁-C₆-alkyl and/or with the group —NR²R³; or for aryl or hetaryl that is optionally substituted in one or more places in the same way or differently with halogen, cyano, hydroxy, C₁-C₆-alkyloxy, C₂-C₆-alkenyl, aryl-C₁-C₆-alkyloxy, aralkyloxy, C₁-C₆-alkyl, halo-C₁-C₆-alkyl or with the group ═O, —SO₂R⁴, OR⁵, —R⁵ or —PO(OR¹²)(OR¹³),

R² and R³, independently of one another, stand for hydrogen or for C₁-C₆-alkyl, C₃-C₆-cycloalkyl, C₃-C₆-cycloalkenyl, aryl or hetaryl that is optionally substituted in one or more places in the same way or differently with halogen, cyano, C₁-C₆-alkyl, phenyl, hydroxy-C₁-C₆-alkyl, halo-C₁-C₆-alkyl, or with the group —NR⁶R⁷, —OR⁵, C₁-C₆-alkyl-OR⁵, —SR⁴, —SOR⁴ or —SO₂R⁴, or

R² and R³, together with the nitrogen atom, form a C₃-C₈-ring, which optionally can contain another nitrogen, sulfur or oxygen atom in the ring, or can contain the group —N(R¹⁰), and which optionally can be substituted in one or more places in the same way or differently with halogen, cyano, C₁-C₆-alkyl, halo-C₁-C₆-alkyl, aryl or with the group —OR⁵, —SR⁴, —SOR⁴ or —SO₂R⁴,

R⁴ stands for hydroxy, C₁-C₆-alkyl, aryl, heteroaryl or for the group —NR²R³,

R⁵ stands for hydrogen, C₁-C₁₋₂-alkyl, halo-C₁-C₆-alkyl, C₃-C₆-cycloalkyl or halo-C₃-C₆-cycloalkyl, or for C₁-C₁₂-alkyl, which is interrupted in one or more places with oxygen or stands for the group —(CH₂)₂NR²R³, —CH₂CN or —CH₂CF₃,

R⁶ and R⁷, independently of one another, stand for hydrogen or C₁-C₆-alkyl, or

R¹ and R⁷ together form a 5- to 7-membered ring that can contain an oxygen or sulfur atom or the group —N(R¹⁰)—,

R⁸ stands for hydrogen or for C₁-C₆-alkyl, C₁-C₆-alkoxy, benzyl, aryl or hetaryl that is optionally substituted with halogen in one or more places,

R⁹ stands for hydrogen, C₁-C₆-alkyl, tri-C₁₋₆-alkylsilyl, aryl, hetaryl or for the group —COR¹¹,

R¹⁰ stands for hydrogen, C₁-C₆-alkyl or aryl,

R¹¹ stands for hydrogen, C₁-C₆-alkyl or for the group —NR²R³, and

R¹² and R¹³, independently of one another, stand for hydrogen or C₁-C₆-alkyl, as well as isomers, enantiomers and salts thereof, overcome the above-indicated drawbacks.

The compounds according to the invention prevent a tyrosine phosphorylation or stop persistent angiogenesis and thus the growth and propagation of tumors, whereby they are distinguished in particular by a slighter inhibition of isoforms of Cytochrome P 450 (2C9 and 2C19).

Many pharmaceutical agents are degraded via these isoforms. In an inhibition of these isoforms, the plasma level of these pharmaceutical agents increases, which can result in undesirable side effects.

Alkyl is defined in each case as a straight-chain or branched alkyl radical, such as, for example, methyl, ethyl, propyl, isopropyl, butyl, isobutyl, sec-butyl, pentyl, isopentyl or hexyl, heptyl, octyl, nonyl, decyl, undecyl, or dodecyl.

Alkoxy is defined in each case as a straight-chain or branched alkoxy radical, such as, for example, methyloxy, ethyloxy, propyloxy, isopropyloxy, butyloxy, isobutyloxy, sec-butyloxy, pentyloxy, isopentyloxy, hexyloxy, heptyloxy, octyloxy, nonyloxy, decyloxy, undecyloxy or dodecyloxy.

Cycloalkyls are defined as monocyclic alkyl rings, such as cyclopropyl, cyclobutyl, cyclopentyl, cyclohexyl, or cycloheptyl, cyclooctyl, cyclononyl or cyclodecyl, but also bicyclic rings or tricyclic rings, such as, for example, adamantanyl.

Cycloalkenyl is defined in each case as cyclobutenyl, cyclopentenyl, cyclohexenyl, cycloheptenyl, cyclooctenyl, cyclononenyl or cyclodecenyl, whereby the linkage can be carried out both to the double bond and to the single bonds.

Halogen is defined in each case as fluorine, chlorine, bromine or iodine.

Alkenyl is defined in each case as a straight-chain or branched alkenyl radical that contains 2-6, preferably 2-4, C atoms. For example, the following radicals can be mentioned: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, but-3-en-1-yl, and allyl.

The aryl radical in each case has 6-12 carbon atoms, such as, for example, naphthyl, biphenyl and especially phenyl.

The heteroaryl radical in each case comprises 3-16 ring atoms, and instead of the carbon can contain one or more heteroatoms that are the same or different, such as oxygen, nitrogen or sulfur, in the ring, and can be monocyclic, bicyclic, or tricyclic, and in addition in each case can be benzocondensed.

For example, there can be mentioned:

Thienyl, furanyl, pyrrolyl, oxazolyl, thiazolyl, imidazolyl, pyrazolyl, isoxazolyl, isothiazolyl, oxadiazolyl, triazolyl, thiadiazolyl, etc., and benzo derivatives thereof, such as, e.g., benzofuranyl, benzothienyl, benzoxazolyl, benzimidazolyl, indazolyl, indolyl, isoindolyl, etc.; or pyridyl, pyridazinyl, pyrimidinyl, pyrazinyl, triazinyl, etc., and benzo derivatives thereof, such as, e.g., quinolyl, isoquinolyl, etc.; or azocinyl, indolizinyl, purinyl, etc., and benzo derivatives thereof; or cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, or oxepinyl, etc.

The aryl radical and the heteroaryl radical in each case can be substituted in the same way or differently in 1, 2 or 3 places with hydroxy, halogen, C₁-C₄-alkoxy, with C₁-C₄-alkyl or C₁-C₄-alkyl that is substituted in one or more places with halogen.

If an acid group is included, the physiologically compatible salts of organic and inorganic bases are suitable as salts, such as, for example, the readily soluble alkali salts and alkaline-earth salts as well as N-methyl-glucamine, dimethyl-glucamine, ethylglucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol.

If a basic group is included, the physiologically compatible salts of organic and inorganic acids are suitable, such as hydrochloric acid, sulfuric acid, phosphoric acid, citric acid, tartaric acid, fumaric acid, i.a.

The compounds of general formula I according to the invention also contain the possible tautomeric forms and comprise the E-isomers or Z-isomers, or, if a chiral center is present, also the racemates and enantiomers.

Those compounds of general formula I in which

A, B, and D, independently of one another, stand for a nitrogen or carbon atom, whereby at least one nitrogen atom is contained in the ring,

E stands for aryl or hetaryl that is optionally substituted in one or more places in the same way or differently with halogen, cyano, C₁-C₆-alky, C₁-C₆-alkoxy, halo-C₁-C₆-alkyl or with the group —OR⁵, —SR⁴, —SOR⁴ or —SO₂R⁴, or for the group —COOR⁸, —CONR²R³, —SR⁴, —SOR⁴, —SO₂R⁴, —SCN, —PO(OR¹²)(OR¹³), —CH═CH—COR⁹ or —C≡C—R⁹,

G stands for a nitrogen atom or for the group —C—X,

L stands for a nitrogen atom or for the group —C—X,

M stands for a nitrogen atom or for the group —C—X,

Q stands for a nitrogen atom or for the group —C—X, whereby at most one nitrogen atom is in the ring,

X stands for hydrogen, halogen or for C₁-C₆-alkyl, C₁-C₆-alkyloxy or C₁-C₆-carboxyalkyl that is unsubstituted or that is optionally substituted in one or more places with halogen,

R¹ stands for aryl or hetaryl that is optionally substituted in one or more places in the same way or differently with halogen, cyano, hydroxy, C₁-C₆-alkyloxy, C₂-C₆-alkenyl, aryl-C₁-C₆-alkyloxy, aralkyloxy, C₁-C₆-alkyl, halo-C₁-C₆-alkyl or with the group ═O, —SO₂R⁴, OR⁵, —R⁵ or —PO(OR¹²)(OR¹³),

R² and R³, independently of one another, stand for hydrogen or for C₁-C₆-alkyl, C₃-C₆-cycloalkyl, C₃-C₆-cycloalkenyl, aryl or hetaryl that is optionally substituted in one or more places in the same way or differently with halogen, cyano, C₁-C₆-alkyl, phenyl, hydroxy-C₁-C₆-alkyl, halo-C₁-C₆-alkyl or with the group —NR⁶R⁷, —OR⁵, C₁-C₆-alkyl-OR⁵, —SR⁴, —SOR⁴ or —SO₂R⁴, or

R² and R³ together with the nitrogen atom form a C₃-C₈ ring, which optionally can contain another nitrogen, sulfur or oxygen atom in the ring, or can contain the group —N(R¹⁰), and which optionally can be substituted in one or more places in the same way or differently with halogen, cyano, C₁-C₆-alkyl, halo-C₁-C₆-alkyl, aryl or with the group —OR⁵, —SR⁴, —SOR⁴ or —SO₂R⁴,

R⁴ stands for hydroxy, C₁-C₆-alkyl, aryl, heteroaryl or for the group —NR²R³,

R⁵ stands for hydrogen, C₁-C₁₋₂-alkyl, halo-C₁-C₆-alkyl, C₃-C₆-cycloalkyl or halo-C₃-C₆-cycloalkyl, or for C₁-C₁₂-alkyl, which is interrupted in one or more places with oxygen, or stands for the group —(CH₂)₂NR²R³, —CH₂CN or —CH₂CF₃,

R⁶ and R⁷, independently of one another, stand for hydrogen or C₁-C₆-alkyl, or

R⁶ and R⁷ together form a 5- to 7-membered ring, which can contain an oxygen or sulfur atom or the group —N(R¹⁰)—,

R⁸ stands for hydrogen or for C₁-C₆-alkyl, C₁-C₆-alkoxy, benzyl, aryl or hetaryl that is optionally substituted with halogen in one or more places,

R⁹ stands for hydrogen, C₁-C₆-alkyl, tri-C₁-C₆-alkylsilyl, aryl, hetaryl or for the group —COR¹¹,

R¹⁰ stands for hydrogen, C₁-C₆-alkyl or aryl,

R¹¹ stands for hydrogen, C₁-C₆-alkyl or for the group —NR²R³, and

R¹² and R¹³, independently of one another, stand for hydrogen or C₁-C₆-alkyl, as well as isomers, enantiomers and salts thereof,

have proven especially effective.

Those compounds of general formula I, in which

A, B and D, independently of one another, stand for a nitrogen or carbon atom, whereby at least one nitrogen atom is contained in the ring,

E stands for aryl or hetaryl that is optionally substituted in one or more places in the same way or differently with halogen, cyano, C₁-C₆alkyl, C₁-C₆alkoxy, halo-C₁-C₆alkyl or with the group —OR⁵, —SR⁴, —SOR⁴ or —SO₂R⁴, or for the group —COOR⁸, —CONR²R³, —SR⁴, —SOR⁴, —SO₂R⁴, —SCN, —PO(OR¹²)(OR¹³), —CH═CH—COR⁹ or —C≡C—R⁹,

G stands for a nitrogen atom or for the group —C—X,

L stands for a nitrogen atom or for the group —C—X,

M stands for a nitrogen atom or for the group —C—X,

Q stands for a nitrogen atom or for the group —C—X, whereby at most one nitrogen atom is in the ring,

X stands for hydrogen or halogen,

R¹ stands for aryl or hetaryl that is optionally substituted in one or more places in the same way or differently with halogen, hydroxy, C₁-C₆-alkyloxy, aralkyloxy, C₁-C₆-alkyl, halo-C₁-C₆-alkyl or with the group —SO₂R⁴, OR⁵, —R⁵ or —PO(OR¹²)(OR¹³),

R² and R³, independently of one another, stand for hydrogen or for C₁-C₆-alkyl, C₃-C₆-cycloalkyl, C₃-C₆-cycloalkenyl, aryl or hetaryl that is optionally substituted in one or more places in the same way or differently with halogen, cyano, C₁-C₆-alkyl, phenyl, hydroxy-C₁-C₆-alkyl, halo-C₁-C₆-alkyl or with the group —NR⁶R⁷, —OR⁵, C₁-C₆-alkyl-OR⁵, —SR⁴, —SOR⁴ or —SO₂R⁴, or

R² and R³ together with the nitrogen atom form a C₃-C₈-ring, which optionally can contain another nitrogen, sulfur or oxygen atom in the ring, or can contain the group —N(R¹⁰), and which optionally can be substituted in one or more places in the same way or differently with halogen, cyano, C₁-C₆-alkyl, halo-C₁-C₆-alkyl, aryl or with the group —OR⁵, —SR⁴, —SOR⁴ or —SO₂R⁴,

R⁴ stands for hydroxy or for the group —NR²R³,

R⁵ stands for hydrogen, C₁-C₁₂-alkyl or for C₁-C₁₂-alkyl, which is interrupted in one or more places with oxygen or stands for the group —(CH₂)₂NR²R³, —CH₂CN or —CH₂CF₃,

R⁶ and R⁷, independently of one another, stand for hydrogen or C₁-C₆-alkyl, or

R⁶ and R⁷ together form a 5- to 7-membered ring, which can contain an oxygen or sulfur atom or the group —N(R¹⁰)—,

R⁸ stands for hydrogen or for C₁-C₆-alkyl, C₁-C₆-alkoxy, benzyl, aryl or hetaryl that is optionally substituted with halogen in one or more places,

R⁹ stands for hydrogen, C₁-C₆-alkyl, tri-C₁-C₆-alkylsilyl, aryl, hetaryl or for the group —COR¹¹,

R¹⁰ stands for hydrogen, C₁-C₆-alkyl or aryl,

R¹¹ stands for hydrogen, C₁-C₆-alkyl or for the group —NR²R³, and

R¹² and R¹³, independently of one another, stand for hydrogen or C₃-C₆-alkyl, as well as isomers, enantiomers and salts thereof,

are especially effective.

[Key: oder=or]

in which T stands for hydrogen, C₁-C₆-alkyl or C₁-C₆-alkoxy,

R² and R³, independently of one another, stand for hydrogen or for C₁-C₆-alkyl, C₃-C₆-cycloalkyl, C₃-C₆-cycloalkenyl, aryl or hetaryl that is optionally substituted in one or more places in the same way or differently with halogen, cyano, C₁-C₆-alkyl, phenyl, hydroxy-C₁-C₆-alkyl, halo-C₁-C₆-alkyl or with the group —NR⁶R⁷—OR⁵, C₁-C₆-alkyl-OR⁵, —SR⁴, —SOR⁴ or —SO₂R⁴, or

R² and R³, together with the nitrogen atom, form a C₃-C₈-ring, which optionally can contain another nitrogen, sulfur or oxygen atom in the ring, or can contain the group —N(R¹⁰), and which optionally can be substituted in one or more places in the same way or differently with halogen, cyano, C₁-C₆-alkyl, halo-C₁-C₆-alkyl, aryl or with the group —OR⁵, —SR⁴, —SOR⁴ or —SO₂R⁴,

R⁴ stands for hydroxy or for the group —NR²R³,

R⁵ stands for hydrogen, C₁-C₁₋₂-alkyl or for C₁-C₁₂-alkyl, which is interrupted in one or more places with oxygen, or stands for the group —CH₂)₂NR²R³, —CH₂CN, or —CH₂CF₃,

R⁶ and R⁷, independently of one another, stand for hydrogen or C₁-C₆-alkyl, or

R⁶ and R⁷ together form a 5- to 7-membered ring that can contain an oxygen or sulfur atom,

R⁸ stands for hydrogen or for C₁-C₆-alkyl, C₁-C₆-alkoxy, benzyl, aryl or hetaryl

Those compounds of general formula I, in which

A, B and D stand for a nitrogen or carbon atom, whereby at least one nitrogen atom is contained in the ring,

E stands for hetaryl that is optionally substituted in one or more places in the same way or differently with halogen, cyano, C₁₋₆alkyl, C₁-C₆-alkoxy, halo-C₁-C₆-alkyl or with the group —OR⁵, —SR⁴, —SOR⁴ or —SO₂R⁴, or for the group —COOR⁸, —CONR²R³, —SR⁴, —SOR⁴, —SO₂R⁴, —SCN, —PO(OR¹²)(OR¹³), —CH═CH—COR⁹ or —C≡C—R⁹,

G stands for the group —C—X,

L stands for the group —C—X,

M stands for the group —C—X,

Q stands for a nitrogen atom or for the group —C—X,

X stands for hydrogen or halogen,

R¹ stands for phenyl, thiophene, furan, oxazole, thiazole, imidazole, pyrazole, pyridine, pyrimidine, triazine, quinoline, or isoquinoline that is optionally substituted in one or more places in the same way or differently with halogen, hydroxy, C₁-C₆-alkyloxy, aralkyloxy, C₁-C₆-alkyl, halo-C₁-C₆-alkyl or with the group —SO₂R⁴, OR⁵, —R⁵ or —PO(OR¹²)(OR¹³) or is substituted on the group

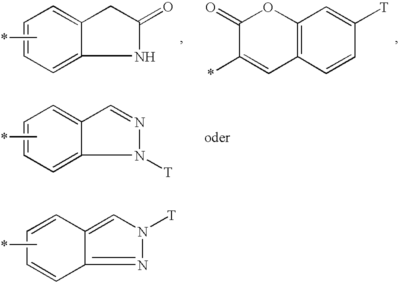

that is optionally substituted in one or more places with halogen, and

R⁹ stands for hydrogen, C₁-C₆-alkyl or tri-C₁-C₆-alkylsilyl, and

R¹² and R¹³, independently of one another, stand for hydrogen or C₁-C₆-alkyl, as well as isomers, enantiomers and salts thereof,

have good properties.

Those compounds of general formula I, in which

A, B and D, independently of one another, stand for a nitrogen or carbon atom, whereby at least one nitrogen atom is contained in the ring,

E stands for thienyl, pyridyl or for the group —COOR⁸, —CONR²R³, or —C≡C—R⁹,

G stands for the group —C—X,

L stands for the group —C—X,

M stands for the group —C—X,

Q stands for a nitrogen atom or for the group —C—X,

X stands for hydrogen or halogen,

R¹ stands for phenyl, thiophene, furan, oxazole, thiazole, imidazole, pyrazole, pyridine, pyrimidine, triazine, quinoline or isoquinoline that is optionally substituted in one or more places in the same way or differently with halogen, hydroxy, C₁-C₆-alkyloxy, aralkyloxy, C₁-C₆-alkyl, halo-C₁-C₆-alkyl or with the group —SO₂R⁴, OR⁵, —R⁵ or —PO(OR¹²)(OR¹³) or substituted on the group 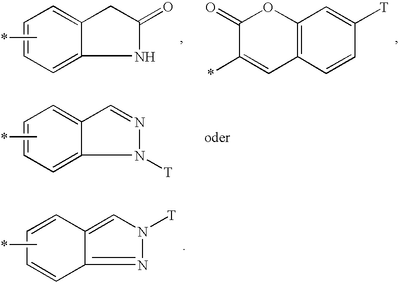 [Key: oder=or

in which T stands for hydrogen, C₁-C₆-alkyl or C₁-C₆-alkoxy,

R² and R³, independently of one another, stand for hydrogen or for C₁-C₆-alkyl, C₃-C₆-cycloalkyl, phenyl or pyridyl that is optionally substituted in one or more places in the same way or differently with halogen, C₁-C₆-alkyl, phenyl or with the group —NR⁶R⁷, —OR⁵ or C₁-C₆-alkyl-OR⁵, or

R² and R³ together with the nitrogen atom form a C₃-C₈-ring, which optionally can contain another nitrogen or oxygen atom in the ring, and which optionally can be substituted in one or more places in the same way or differently with C₁-C₆-alkyl,

R⁴ stands for hydroxy or for the group —NR³R³,

R⁵, R⁶ and R⁷, independently of one another, stand for hydrogen or C₁-C₆-alkyl, or

R⁶ and R⁷ together form a 5- to 7-membered ring, which can contain an oxygen or sulfur atom,

R⁸ stands for hydrogen, C₁-C₆-alkyl or benzyl, and

R⁹ stands for hydrogen, C₁-C₆-alkyl or tri-C₁-C₆-alkylsilyl, and

R¹² and R³, independently of one another, stand for hydrogen or C—C₆-alkyl, as well as isomers and salts thereof,

have excellent properties.

The compounds according to the invention as well as their physiologically compatible salts prevent a tyrosine phosphorylation or stop the persistent angiogenesis and thus the growth and a propagation of tumors, whereby they are distinguished in particular by a slighter inhibition of isoforms of Cytochrome P 450 (2C9 and 2C19). Medication using the compounds according to the invention can therefore be done at no risk even without regard to pharmaceutical agents that are administered at the same time and that are degraded via these isoforms.

The compounds of formula I as well as their physiologically compatible salts can be used as pharmaceutical agents based on their inhibitory activity relative to the phosphorylation of the VEGF receptor. Based on their profile of action, the compounds according to the invention are suitable for treating diseases that are caused or promoted by persistent angiogenesis.

Since the compounds of formula I are identified as inhibitors of the tyrosine kinases VEGFR-1 and VEGFR-2, they are suitable in particular for treating those diseases that are caused or promoted by persistent angiogenesis that is triggered via the VEGF receptor or by an increase in vascular permeability.

The subject of this invention is also the use of the compounds according to the invention as inhibitors of the tyrosine kinases VEGFR-1 and VEGFR-2, or KDR and FLT.

Subjects of this invention are thus also pharmaceutical agents for treating tumors or use thereof.

The compounds according to the invention can be used either alone or in a formulation as pharmaceutical agents for treating psoriasis, Kaposi's sarcoma, restenosis, such as, e.g., stent-induced restenosis, endometriosis, Crohn's disease, Hodgkin's disease, leukemia; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, and for inhibiting the reocclusion of vessels after balloon catheter treatment, in vascular prosthetics or after mechanical devices are used to keep vessels open, such as, e.g., stents, as immunosuppressive agents, for supporting scar-free healing, in senile keratosis and in contact dermatitis.

In treating injuries to nerve tissue, quick scar formation on the injury sites can be prevented with the compounds according to the invention, i.e., scar formation is prevented from occurring before the axons reconnect. A reconstruction of the nerve compounds was thus facilitated.

The formation of ascites in patients can also be suppressed with the compounds according to the invention. VEGF-induced edemas can also be suppressed.

Lymphangiogenesis plays an important role in lymphogenic metastasizing (Karpanen, T. et al., Cancere Res. 2001 Mar 1, 61(5): 1786-90, Veikkola, T., et al., EMBO J. 2001, Mar 15; 20 (6): 1223-31).

The compounds according to the invention now also show excellent action as VEGFR kinase 3 inhibitors and are therefore also suitable as effective inhibitors of lymphangiogenesis.

By a treatment with the compounds according to the invention, not only a reduction in the size of metastases but also a reduction in the number of metastases is achieved.

The compounds according to the invention are also effective in the case of diseases that are associated with excessive lymphangiogenesis and are therefore expected in the lymphangiohyperplasia and—dysplasia syndrome.

Such pharmaceutical agents, their formulations and uses, are also subjects of this invention.

The invention thus also relates to the use of the compounds of general formula I for the production of a pharmaceutical agent for use as or for treatment of psoriasis, Kaposi's sarcoma, restenosis, such as, e.g., stent-induced restenosis, endometriosis, Crohn's disease, Hodgkin's disease, leukemia; arthritis, such as rheumatoid arthritis, hemangioma, angiofibroma; eye diseases, such as diabetic retinopathy, neovascular glaucoma; renal diseases, such as glomerulonephritis, diabetic nephropathy-, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, and for inhibiting the reocclusion of vessels after balloon catheter treatment, in vascular prosthetics or after mechanical devices are used to keep vessels open, such as, e.g., stents, as immunosuppressive agents, for supporting scar-free healing, in senile keratosis and in contact dermatitis.

The formation of ascites in patients can also be suppressed with the compounds according to the invention. VEGF-induced edemas can also be suppressed.

To use the compounds of formula I as pharmaceutical agents, the latter are brought into the form of a pharmaceutical preparation, which in addition to the active ingredient for enteral or parenteral administration contains suitable pharmaceutical, organic or inorganic inert carrier materials, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, capsules or in liquid form, for example as solutions, suspensions or emulsions. They also contain, moreover, adjuvants such as preservatives, stabilizers, wetting agents or emulsifiers, salts for changing osmotic pressure or buffers.

For parenteral administration, especially injection solutions or suspensions, especially aqueous solutions of the active compounds in polyhydroxyethoxylated castor oil, are suitable.

As carrier systems, surface-active adjuvants such as salts of bile acids or animal or plant phospholipids, but also mixtures thereof as well as liposomes or components thereof can also be used.

For oral administration, especially tablets, coated tablets or capsules with talc and/or hydrocarbon vehicles or binders, such as for example, lactose, corn starch or potato starch, are suitable. The administration can also be carried out in liquid form, such as, for example, as juice, to which optionally a sweetener or, if necessary, one or more flavoring substances, is added.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into 2 or more daily doses.

The above-described formulations and forms for dispensing are also subjects of this invention.

The production of the compounds according to the invention is carried out according to methods that are known in the art. For example, compounds of formula I are obtained, in that a compound of general formula II

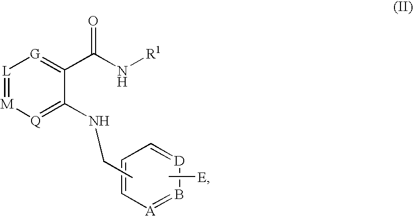

in which A, B, D, G, L, M, Q, W and R¹ have the meanings that are indicated in general formula I and E stands for a carboxylic acid —COOH,

is reacted in a suitable solvent and a suitable organic base, with an amine of general formula III

H—NR⁸R⁹ (III),

in which R⁸ and R⁹ have the meanings that are indicated in general formula I, according to processes that are known in the literature, or if E means a nitrile group, the nitrile is saponified to form amide, or a compound of general formula IV

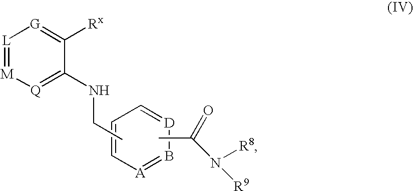

in which A, B, D, G, L, M, Q, W, R⁸ and R⁹ have the meanings that are indicated in general formula I, and R^{x} means an ester or acid group, is converted into the corresponding amide.

The amide formation is carried out according to methods that are known in the literature.

For amide formation, it is possible to start from a corresponding ester. The ester is reacted according to J. Org. Chem. 1995, 8414 with aluminum trimethyl and the corresponding amine in solvents such as toluene at temperatures of 0° C. to the boiling point of the solvent. If the molecule contains two ester groups, both are converted into the same amide. Instead of aluminum trimethyl, sodium hexamethyldisilazide can also be used.

For amide formation, however, all processes that are known from peptide chemistry are also available. For example, the corresponding acid can be reacted with the amine in aprotic polar solvents, such as, for example, dimethylformamide, via an activated acid derivative, obtainable, for example, with hydroxybenzotriazole and a carbodiimide, such as, for example, diisopropylcarbodiimide, or else with preformed reagents, such as, for example, HATU (Chem. Comm. 1994, 201) or BTU, at temperatures of between 0° C. and the boiling point of the solvent, preferably at 80° C. For the amide formation, the process can also be used with the mixed acid anhydride, imidazolide or azide.

Nitriles can also be saponified to form amides according to processes that are known in the literature. The reaction with potassium carbonate and hydrogen peroxide is very effective in an aprotic polar solvent such as dimethyl sulfoxide, preferably at room temperature according to Synthesis, 1989, 949.

In addition, the compounds of general formula I according to the invention can be produced in that a compound of general formula IIa

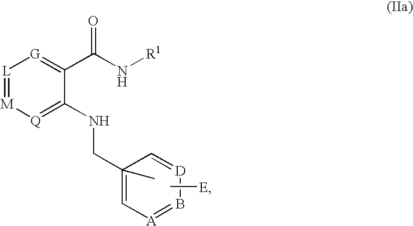

in which A, B, D, G, L, M, Q, W and R¹ have the meanings that are indicated in general formula I, and E means a halogen or an O-sulfonate, such as, e.g., a chlorine, bromine or iodine atom, an O-trifluoromethanesulfonate or O-methylsulfonate,

a. is reacted with appropriately substituted terminal alkenes in a Heck reaction (cf. “Palladium Reagents in Organic Syntheses,” Academic Press 1985, New York, pp. 179 ff.) or with vinylboronic acids or vinylboronic acid esters in a Suzuki reaction (cf. Tetrahedron Lett. 1983, 39, 3271 ff.) or with vinyl stannanes in a Stille reaction (cf. Pure & Appl. Chem. 1985, 57, 1771), or

b. is coupled with any substituted terminal alkines, for example, according to the method of Stephens-Castro (cf. J. Org. Chem. 1963, 28, 3313 ff.) or palladium-catalyzed according to the method of Sonogashira (cf. “Comprehensive Organic Synthesis: Carbon-Carbon σ-Bond Formation,” Pergamon Press 1991, Oxford UK, Volume 3, pp. 551ff.), or

c. is coupled with aryl and hetaryl boronic acids or their esters in a Suzuki reaction (cf. Acc. Chem. Res. 1991, 63, 419 ff. or J. Am. Chem. Soc. 2000, 122, 4020 ff.) or with aryl and hetaryl stannanes in a Stille reaction (cf. Angew. Chem. 1986, 98, 504 ff. or Angew. Chem. Int. Ed. 1999, 38, 2411 ff.) or with aryl and hetaryl Grignard compounds or the analogous zinc-organic derivatives in a Negishi reaction (cf. “Metal-Catalyzed Cross-Coupling Reaction,” Eds. Diederich/Stang, Wiley-VCH 1998, New York, Chapter 1 or else J. Am. Chem. Soc. 2001, 123, 2719 ff.), or

d. is converted in a palladium-catalyzed carbonylation under 1 to 20 bar of carbon monoxide atmosphere in dimethylformamide in the presence of the corresponding alcohol (cf. “Palladium Reagents in Organic Syntheses,” Academic Press 1985, New York, pp. 352 ff. or Synth. Comm. 1997, 27, 515 ff.) into the corresponding carboxylic acid ester, or

e. is converted in a palladium-catalyzed carbonylation under 1 to 20 bar of carbon monoxide atmosphere in dimethylformamide-water mixtures into the corresponding carboxylic acid (cf. J. Org. Chem. 1981, 46, 4614 ff.). The carboxylic acids can also be obtained by saponification of the carboxylic acid esters, or

f. the corresponding carboxylic acid amides are produced in a palladium-catalyzed carbonylation under 1 to 20 bar of carbon monoxide atmosphere in dimethylformamide in the presence of amines (cf. “Palladium Reagents in Organic Syntheses,” Academic Press 1985, New York, pp. 352 ff., Tetrahedron Lett. 1982, 23, 3383 ff.). The synthesis of the carboxylic acid amides can also be carried out from carboxylic acid esters; the method according to Weinreb has especially proven its value here (cf. Tetrahedron Lett. 1977, 17, 4171 ff., J. Org. Chem. 1995, 60, 8414 ff.). The carboxylic acid amides can also be synthesized from the carboxylic acids that are produced under e); basically all processes that are known from the peptide chemistry are available for this purpose (cf. Synthesis 1972, 453-63 or “Comprehensive Organic Transformations,” Wiley-VCH 1989, New York, 972-6). For example, the corresponding carboxylic acid in aprotic polar solvents, such as, for example dimethylformamide, can be reacted with an activated carboxylic acid derivative, produced, for example, by adding carbonyldiimidazole, at temperatures of between 0-120° C., preferably at room temperature, with amines, such as, for example HATU (Chem. Comm. 1994, 201), or

g. the corresponding sulfide is converted with thioalkylene, thioarylene and thiohetarylene directly, in the presence of bases, such as, for example, potassium hydride or potassium tert-butanolate or transition metals, such as, for example, copper chips, copper chloride or copper bromide or palladium dichloride in aprotic solvents, such as, for example, dimethylformamide, N-methylpyrrolidone, dimethyl sulfoxide or xylene at temperatures of between 20-200° C. The execution of the reaction in a microwave device can turn out to be advantageous in this case (cf. Tetrahedron 1983, 39, 4153 ff.). The production of 2-thio-substituted pyridyl derivatives can also be carried out easily from the 2-pyridone derivative after thionylation with phosphorus pentasulfide (cf. Bull. Soc. Chim. Fr.; 1953; 1001 ff.) or Lawesson 's reagent (Tetrahedron 1984, 40, 2047 ff.) and subsequent alkylation with alkyl halides, preferably with alkyl iodides (cf. J. Org. Chem. 1999; 64, 7935-9) or alkyl sulfonates, preferably alkyltrifluoromethylsulfonates.

h. The corresponding sulfoxides can be obtained by oxidation of sulfides with standard oxidizing agents, such as, for example, hydrogen peroxide, sodium periodate, tert-butoxy hypochlorite, sodium chlorite, metachloroperbenzoic acid, trifluoroperoxyacetic acid, dimethyl dioxiram, cerium ammonium nitrate or nitric acid (cf. “Oxidations in Organic Chemistry,” ACS Washington 1990, pp. 252-63) in solvents, such as, for example, dichloromethane, dichloroethane, chloroform, tetrahydrofuran, acetonitrile, dimethylformamide, N-methylpyrrolidinone, dimethyl sulfoxide, dimethoxyethane, diglyme, tetraglyme or water, at temperatures of between 20° C. and the boiling point of the solvent. The thus obtained sulfoxides can further be oxidized to the corresponding sulfones; the latter is achieved, for example, by oxidizing agents such as hydrogen peroxide, potassium permanganate, sodium perborate or potassium hydrogen persulfate (cf. Tetrahedron Lett. 1981, 22, 1287 ff.) in solvents, such as, for example, dichloromethane, dichloroethane, chloroform, tetrahydrofuran, acetonitrile, dimethylformamide, N,N-dimethylacetamide, dimethyl sulfoxide or water, at temperatures of between 20° C. and the boiling point of the solvent. The treatment of sulfides with an excess of the above-cited oxidizing agents results directly in the corresponding sulfones (cf. “The Chemistry of Sulphones and Sulfoxides” in Patai, Wiley 1988, New York, pp. 165-231).

i. By oxidation of the thiols that are obtained under g), the chlorosulfonates can be produced; the oxidation with chlorine in aqueous hydrochloric acid (cf. J. Org. Chem. 1999; 64, 5896-903) or carbon tetrachloride (cf. J. Med. Chem. 2000, 43, 843-58) or with sodium hypochlorite in sulfuric acid (cf. Tetrahedron Asymm. 1997; 8; 3559-62) has especially proven its value here.

j. By reaction with a mixture that consists of copper rhodanide and potassium rhodanide in polar aprotic solvents, such as, for example, acetonitrile, dimethylformamide, N,N-dimethylacetamide, dimethyl sulfoxide, diglyme, tetraglyme, N-methylpyrrolidinone, the corresponding thiocyanates can be obtained (cf. J. Chem. Soc. Chem. Comm. 1989, 81 ff). From the latter in turn, the corresponding sulfonic acid chlorides can be obtained by oxidation with hypochlorite.

k. By reaction of the chlorosulfonates that are cited under i) with amines, in solvents, such as, for example, dichloromethane, dichloroethane, chloroform, tetrahydrofuran, ethyl acetate, acetonitrile, dimethylformamide, N-methyl-pyrrolidone, N,N-dimethylacetamide, dimethoxyethane or water, at temperatures of between 0° C. and the boiling point of the solvent, the corresponding sulfonamides can be obtained (cf. Tetrahedron 2000, 56, 8253-62).

l. By hydrolysis of the chlorosulfonates that are obtained under i) in water or aqueous alkaline solution at temperatures of between 5° C. and 100° C., the corresponding sulfonic acids are obtained.

m. By palladium-catalyzed reaction with O,O-dialkylphosphites in aprotic solvents, such as, for example, dimethylformamide, N-methyl-pyrrolidinone, N,N-dimethylacetamide, dimethyl sulfoxide or toluene in the presence of a base, such as, for example, triethylamine or diisopropylethylamine, at temperatures of between 0° C. and the boiling temperature of the solvent, preferably at 80° C., the corresponding phosphonates can be obtained (cf. Bull. Chem. Soc. Jpn. 1982, 55, 909 ff.).

n. By metallation, for example with n-butyllithium, sec-butyllithium, tert-butyllithium, methyllithium, lithium diisopropylamide or ethyl magnesium bromide, in aprotic solvents such as, for example, diethyl ether, tetrahydrofuran or dioxane, at temperatures of between −100° C. and 0° C., preferably at −78° C. in tetrahydrofuran and reaction with isocyanates, the corresponding carboxylic acid amides can be obtained.

o. By having the reaction carried out analogously to what is described under n) and having the recovery of the metallated intermediate stages be done with chloroformic acid ester, the corresponding carboxylic acid esters can be obtained.

p. By having the reaction carried out analogously to what is described under n) and having the recovery of the metallated intermediate stages be done with dimethylformamide, ethyl formate or N-formylmorpholine, the corresponding aldehydes can be obtained.

q. By having the reaction carried out analogously to what is described under n) and having the recovery of the metallated intermediate stages be done with alkyl halides or alkyl sulfonates, preferably alkyl iodides or alkyltrifluoromethanesulfonates, the corresponding pyridylalkyl derivatives can be produced.

r. By reduction with hydrogen in the presence of catalytic amounts of palladium, nickel or rhodium metal or salts of these metals, for example palladium on activated carbon in polar-protic solvents or solvent mixtures, such as, for example, methanol-glacial acetic acid, the pyridylalkenes that are produced under a) and the pyridylalkines that are produced under b) are converted into the corresponding pyridylalkanes.

The sequence of the process steps can also be interchanged in all cases.

Production of the Compounds According to the Invention

The following examples explain the production of the compounds according to the invention without the scope of the claimed compounds being limited to these examples.

### EXAMPLE 1

Production of 5-{[2-(Isoquinolin-3-ylcarbamoyl)-phenylamino]-methyl}-pyridine-2-carboxylic acid propylamide

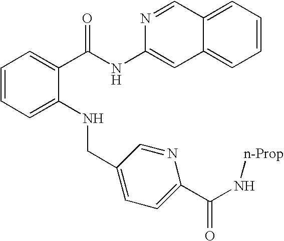

50 mg (0.13 mmol) of 5-{[2-(isoquinolin-3-ylcarbamoyl)-phenylamino]-methyl}-pyridine-2-carboxylic acid and 42 mg (0.26 mmol) of carbonyldiimidazole are introduced into 2.5 ml of dimethylformamide under argon and in a moisture-free environment, and it is stirred for 30 minutes at room temperature. 15 mg (0.26 mmol) of n-propylamine is then added to the batch, and stirring is continued for 12 hours at room temperature. It is then diluted with water to about 30 ml and shaken out three times with 20 ml of ethyl acetate each. The collected organic phase is dried, filtered and concentrated by evaporation, and the residue is chromatographed on a Flash column (5 g; Isolute flash silica, Separtis Company) with a gradient of 100% hexane to 50% hexane and 50% ethyl acetate. 45 mg (79% of theory) of 5-{[2-(isoquinolin-3-ylcarbamoyl)-phenylamino]-methyl}-pyridine-2-carboxylic acid propylamide with a molar peak in MS m/e=439 is obtained.

Similarly produced are also the following compounds:

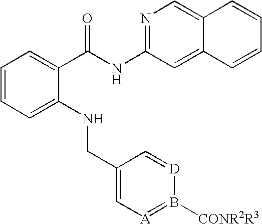

| | | | | | | | Smp. [° C.]/MS |
|---|---|---|---|---|---|---|---|
| Beispiel Nr. | A | B | D | R² | R³ | MW | Molpeak (m/e) |
| 1.1 | C | C | N | —CH₃ | —CH₃ | 425.49 | |
| 1.2 | C | C | N | —CH(CH₃)₂ | H | 439.52 | Harz/439 |
| 1.3 | C | C | N | 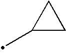 | H | 437.50 | Harz/437 |
| 1.4 | C | C | N | —CH₂CF₃ | H | | |
| 1.5 | C | C | N | —(CH₂)₂-OH | H | 441.49 | Harz/441 |
| 1.6 | C | C | N | —(CH₂)₃OH | H | 455.52 | Harz/455 |
| 1.7 | C | C | N | —(CH₂)₄OH | H | 469.54 | Harz/469 |
| 1.8 | C | C | N | 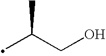 | H | 455.52 | 155 |
| 1.9 | C | C | N | 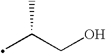 | H | 455.52 | Harz/455 |
| 1.10 | C | C | N | 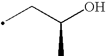 | H | 455.52 | 109 |
| 1.11 | C | C | N | 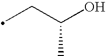 | H | 455.52 | 82 |
| 1.12 | C | C | N | 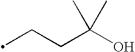 | H | 483.87 | Harz/483 |
| 1.13 | C | C | N | —(CH₂)₂N(CH₃)₂ | H | 468. | /468 |
| 1.14 | C | C | N | —(CH₂)₃N(CH₃)₂ | H | 482.59 | /469 |

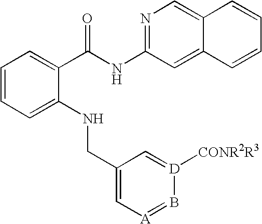

| | | | | | | | Smp. [° C.]/MS |
|---|---|---|---|---|---|---|---|
| Beispiel Nr. | A | B | D | R² | R³ | MW | Molpeak (m/e) |
| 1.15 | C | N | C | —CH₃ | —CH₃ | 425.49 | 106 |
| 1.16 | C | N | C | —CH₃ | H | 411.46 | 180 |
| 1.17 | C | N | C | —C₂H₅ | H | 425.49 | 165 |
| 1.18 | C | N | C | 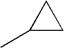 | H | 437.50 | 172 |
| 1.19 | C | N | C | —(CH₂)₂—OH | H | 441.49 | 136 |
| 1.20 | C | N | C | 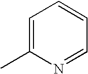 | H | 474.52 | 207 |
| 1.21 | C | N | C | 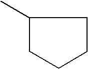 | H | 465.55 | 94 |
| 1.22 | C | N | C | 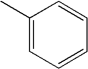 | H | 473.53 | 187 |
| 1.23 | C | N | C | —C₃H₇ | H | 439.52 | 96 |
| 1.24 | C | N | C | —CH(CH₃)₂ | H | 439.52 | 174 |
| 1.25 | C | N | C | 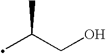 | H | 455.52 | 103 |
| 1.26 | C | N | C | 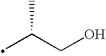 | H | 455.52 | 110 |
| 1.27 | C | N | C | 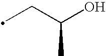 | H | 455.52 | 105 |
| 1.28 | C | N | C | 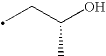 | H | 455.52 | 100 |
| 1.29 | C | N | C | 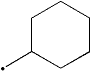 | H | 479.58 | 110 |
| 1.30 | C | N | C | 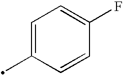 | H | 491.52 | 204 |
| 1.31 | C | N | C | 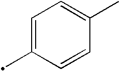 | H | 487.56 | 151 |
| 1.32 | C | N | C | —(CH₂)₃—OH | H | 455.52 | 65 |
| 1.33 | C | N | C | —(CH₂)₅—OH | H | 483.57 | 70 |
| 1.34 | C | N | C | —(CH₂)₄—OH | H | 469.54 | 70 |
| 1.35 | C | N | C | —(CH₂)₂N(CH₃)₂ | H | 455.52 | 98 |
| 1.36 | C | N | C | —(CH₂)₃N(CH₃)₂ | H | 482.59 | 95 |
| 1.37 | C | N | C | 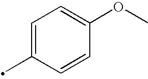 | H | 503.56 | 190 |
| 1.38 | C | N | C | 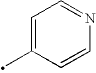 | H | 474.52 | 190 |
| 1.39 | C | N | C | 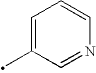 | H | 474.52 | 105 |
| 1.40 | C | N | C | 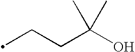 | H | 483.57 | 75 |
| 1.41 | C | N | C | 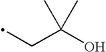 | H | 469.54 | 50 |
| 1.42 | C | N | C | 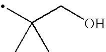 | H | 469.54 | 170 |
| 1.43 | C | N | C | —(CH₂)₂OCH₃ | H | 455.52 | 67 |
| 1.44 | C | N | C | 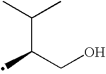 | H | 483.57 | 86 |
| 1.45 | C | N | C | 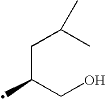 | H | 497.6 | 86 |
| 1.46 | C | N | C | 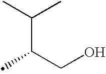 | H | 483.57 | 66 |
| 1.47 | C | N | C | 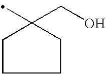 | H | 495.58 | 148 |
| 1.48 | C | N | C | 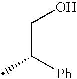 | H | 517.58 | 78 |
| 1.49 | C | N | C | 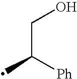 | H | 517.58 | 91 |
| 1.50 | C | N | C | 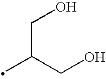 | H | 471.51 | 85 |
| 1.51 | C | N | C | 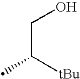 | H | 497.59 | 98 |
| 1.52 | C | N | C | CH₂CF₃ | H | 479.46 | 96 |
| 1.53 | C | N | C | 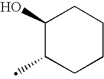 | H | 495.58 | 127 |
| 1.54 | C | N | C | 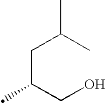 | H | 497.59 | 96 |
| 1.55 | C | N | C | 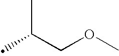 | H | 469.54 | 78 |
| 1.56 | C | N | C | 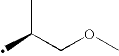 | H | 469.54 | 78 |
| 1.57 | C | N | C | 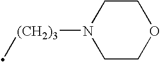 | H | 510.59 | |
| 1.58 | C | N | C | 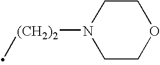 | H | 524.62 | |
| 1.59 | C | N | C | 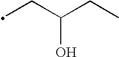 | H | 469.54 | |

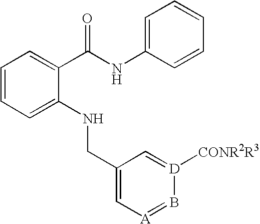

| | | | | | | | Smp. |
|---|---|---|---|---|---|---|---|
| | | | | | | | [° C.] |
| | | | | | | | oder MS |
| Beispiel | | | | | | | Molpeak |
| Nr. | A | D | B | R² | R³ | MW | (m/e) |
| 1.60 | C | C | N | —CH(CH₂OH)₂ | H | 488,46 | 97 |
| 1.61 | C | C | N | —(CH₂)₃OH | H | 500.52 | 125 |
| 1.62 | C | C | N | —(CH₂)₂—OMe | H | 472.46 | 67 |
| 1.63 | C | C | N | —(CH₂)₅OH | H | 500.52 | 92 |
| 1.64 | C | C | N | —(CH₂)₄OH | H | 486.49 | 73 |
| 1.65 | C | C | N | 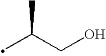 | H | 472.46 | 82 |
| 1.66 | C | C | N | 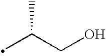 | H | 472.46 | 73 |
| 1.67 | C | C | N | 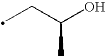 | H | 472.46 | 87 |
| 1.68 | C | C | N | 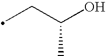 | H | 472.46 | 93 |
| 1.69 | C | C | N | 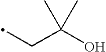 | H | 486.49 | 67 |
| 1.70 | C | C | N | 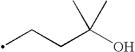 | H | 500.52 | 67 |
| 1.71 | C | C | N | —(CH₂)₂N(CH₃)₂ | H | 485.51 | 82 |
| 1.72 | C | C | N | —(CH₂)₂N(CH₃)₂ | H | 499.53 | 74 |
| 1.73 | C | C | N | 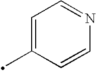 | H | 491.47 | 142 |
| 1.74 | C | C | N | 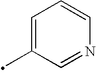 | H | 491.47 | 104 |
| 1.75 | C | C | N | 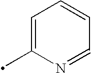 | H | 491.47 | 73 |

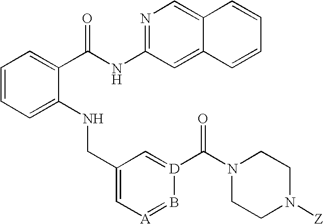

| Beispiel | | | | | | Smp. [° C.]/MS |
|---|---|---|---|---|---|---|
| Nr. | A | B | D | Z | MW | Molpeak (m/e) |
| 1.76 | C | N | C | —CH₃ | 480.57 | 99 |

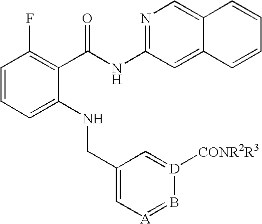

| | | | | | | | |
|---|---|---|---|---|---|---|---|
| | | | | | | | Smp. [° C.]/ |
| Beispiel | | | | | | | MS Molpeak |
| Nr. | A | B | D | R² | R³ | MW | (m/e) |

| | | | | | | |
|---|---|---|---|---|---|---|
| 1.77 | C | N | C | 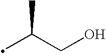 | H | 473.50 |
| 1.78 | C | N | C | 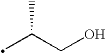 | H | 473.50 |
| 1.79 | C | N | C | 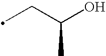 | H | 473.50 |
| 1.80 | C | N | C | 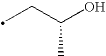 | H | 473.50 |
| 1.81 | C | N | C | —(CH₂)₂N(CH₃)₂ | H | 486.54 |

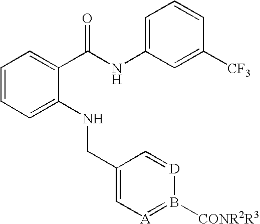

| | | | | | | | Smp. |
|---|---|---|---|---|---|---|---|
| | | | | | | | [° C.] |
| | | | | | | | oder MS |
| Beispiel | | | | | | | Molpeak |
| Nr. | A | B | D | R² | R³ | MW | (m/e) |
| 1.82 | C | C | N | —(CH₂)₃OH | H | 500,52 | 80 |
| 1.83 | C | C | N | 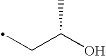 | H | 472.46 | 50 |
| 1.84 | C | C | N | 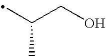 | H | 472.46 | 83 |
| 1.85 | C | C | N | 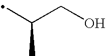 | H | 472.46 | 129 |
| 1.86 | C | C | N | 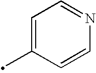 | H | 491.47 | 150 |
| 1.87 | C | C | N | 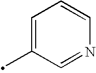 | H | 491.47 | 148 |
| 1.88 | C | C | N | —(CH₂)₅OH | H | 500.52 | 101 |
| 1.89 | C | C | N | —CH(CH₂OH)₂ | H | 488.46 | 144 |
| 1.90 | C | C | N | —(CH₂)₃N(CH₃)₂ | H | 499.53 | 117 |
| 1.91 | C | C | N | —(CH₂)₂—OMe | H | 472.46 | 54 |
| 1.92 | C | C | N | 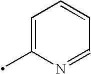 | H | 491.47 | 121 |
| 1.93 | C | C | N | —(CH₂)₂N(CH₃)₂ | H | 485.51 | 139 |
| 1.94 | C | C | N | 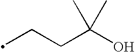 | H | 500.52 | 70 |
| 1.95 | C | C | N | 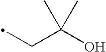 | H | 486.49 | 88 |
| 1.96 | C | C | N | 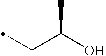 | H | 472.46 | 76 |
| 1.97 | C | C | N | —(CH)₄OH | H | 488.52 | |

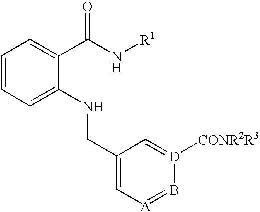

| | | | | | | | | Smp. [° C.]/ |
|---|---|---|---|---|---|---|---|---|
| Beispiel | | | | | | | | MS Molpeak |
| Nr. | A | B | D | R¹ | R² | R³ | MW | (m/e) |
| 1.98 | C | N | C | 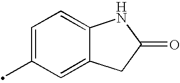 | 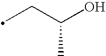 | H | 459.50 | |
| 1.99 | C | N | C | 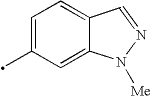 | 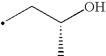 | H | 458.51 | |
| 1.100 | C | N | C | 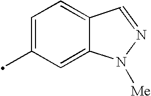 | 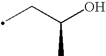 | H | 458.51 | |
| 1.101 | C | N | C | 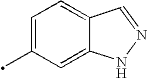 | 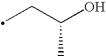 | H | 444.49 | |
| 1.102 | C | N | C | 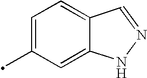 | 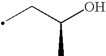 | H | 444.49 | |
| 1.103 | C | N | C | 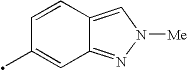 | 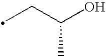 | H | 458.51 | |
| 1.104 | C | N | C | 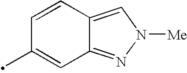 | 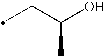 | H | 458.51 | |
| 1.105 | C | N | C | 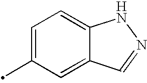 | 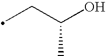 | H | 444.49 | |
| 1.106 | C | N | C | 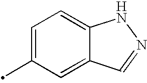 | 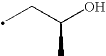 | H | 444.49 | |

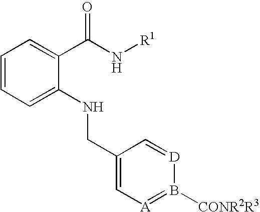

| | | | | | | | | Smp. [° C.]/ |
|---|---|---|---|---|---|---|---|---|
| Beispiel | | | | | | | | MS Molpeak |
| Nr. | A | D | B | R¹ | R² | R³ | MW | (m/e) |
| 1.107 | C | N | C | 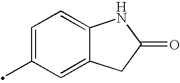 | 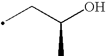 | H | 459.50 | 160.7 |
| 1.108 | C | N | C | 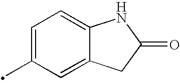 | 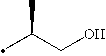 | H | 459.50 | 123.8 |
| 1.109 | C | N | C | 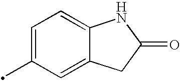 | 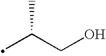 | H | 459.50 | 123 |
| 1.110 | C | N | C | 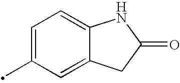 | 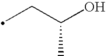 | H | 459.50 | |
| 1.111 | C | N | C | 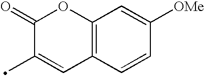 | 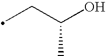 | H | 502.52 | 199.2 |
| 1.112 | C | N | C | 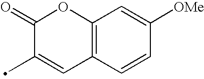 | 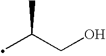 | H | 502.52 | 180.4 |
| 1.113 | C | N | C | 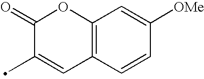 | 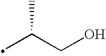 | H | 502.52 | |
| 1.114 | C | N | C | 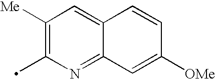 | 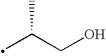 | H | 499.56 | |
| 1.115 | C | N | C | 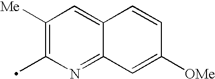 | 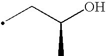 | H | 499.56 | 174 |
| 1.116 | C | N | C | 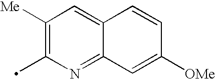 | 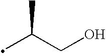 | H | 499.56 | 173.8 |
| 1.117 | C | N | C | 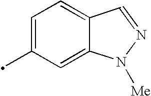 | 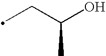 | H | 458.51 | |
| 1.118 | C | N | C | 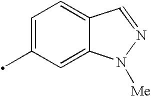 | 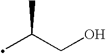 | H | 458.51 | |
| 1.119 | C | N | C | 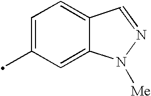 | 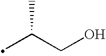 | H | 458.51 | |
| 1.120 | C | N | C | 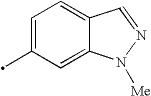 | 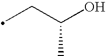 | H | 458.51 | |
| 1.121 | C | N | C | 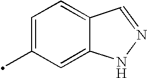 | 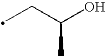 | H | 444.49 | |
| 1.122 | C | N | C | 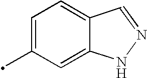 | 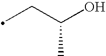 | H | 444.49 | |
| 1.123 | C | N | C | 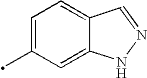 | 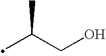 | H | 444.49 | |
| 1.124 | C | N | C | 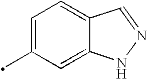 | 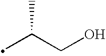 | H | 444.49 | |
| 1.125 | C | N | C | 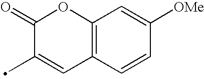 | 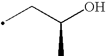 | H | 502.52 | |

### EXAMPLE 2.0

Production of 5-{[2-(Isoquinolin-3-ylcarbamoyl)-phenylamino]-methyl}-pyridine-2-carboxylic Acid Pyridin-3-ylamide

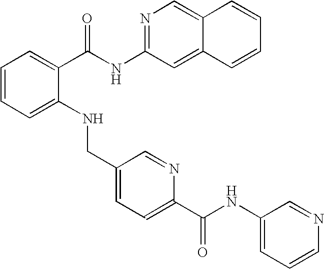

120 mg (0.3 mmol) of 5-{[2-(isoquinolin-3-ylcarbamoyl)-phenylamino]-methyl}-pyridine-2-carboxylic acid is dissolved under argon in 5 ml of absolute dimethylformamide, mixed with 56 mg (0.6 mmol) of 3-aminopyridine, 76 mg (0.75 mmol) of N-methylmorpholine and 136 mg (0.36 mmol) of O-(7-azabenzotriazol-1-yl)-1,1,3,3-tetramethyluronium hexafluorophosphate (HATU) and stirred for 48 hours at room temperature. Then, it is concentrated by evaporation in a vacuum, and the residue is chromatographed on a flash column (5 g of Isolute flash silica, Sepostis Company) with a gradient of methylene chloride: ethanol=100:0 to 95:5 as an eluant. Mg of5-{[2-(isoquinolin-3-ylcarbamoyl)-phenylamino]-methyl}-pyridine-2-carboxylic acid pyridin-3-ylamide is obtained.

MS (m/e 474)

Similarly produced are:

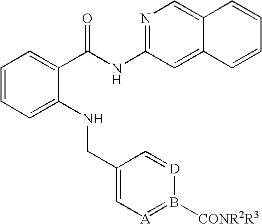

| | | | | | | | Smp. [° C.]/ |
|---|---|---|---|---|---|---|---|
| Beispiel | | | | | | | MS Molpeak |
| Nr. | A | B | D | R² | R³ | MW | (m/e) |
| 2.1 | C | C | N | 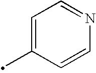 | H | 474.52 | 474 (m/e) |
| 2.2 | C | C | N | 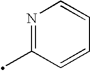 | H | 474.52 | 474 (m/e) |

[Key:]
Beispiel Nr. = Example No.
Smp. = Melting point
Molpeak = Molar peak

### EXAMPLE 3.0

Production of 5-{[2-(2-Oxo-2,3-dihydro-1H-indol-5-ylcarbamoyl)-phenylamino]-methyl}-pyridine-2-carboxylic Acid Amide

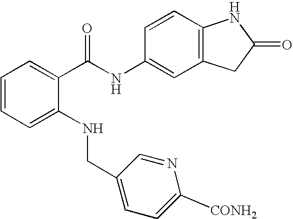

36 mg (0.09 mmol) of 2-[(6-cyano-pyridin-3-ylmethyl)-amino]-N-(2-oxo-2,3-dihydro-1H-indol-5-yl)-benzamide is mixed in 1 ml of dimethyl sulfoxide with 30 mg (0.22 mmol) of potassium carbonate and 0.05 ml (0.42 mmol) of hydrogen peroxide (30%), and it is stirred for 3.5 hours at room temperature. It is then diluted with water and extracted with ethyl acetate. The organic phase is washed, dried, filtered and concentrated by evaporation. The residue is absorptively precipitated with warm methanol. 5 mg (11% of theory) of 5-{[2-(2-oxo-2,3-dihydro-1H-indol-5-ylcarbamoyl)-phenylamino]-methyl}-pyridine-2-carboxylic acid amide is obtained.

Similarly produced are:

### EXAMPLE 3.1

4-{[2-(7-Methoxy-3-methyl-quinolin-2-ylcarbamoyl)-phenylamino]-methyl-pyridine-2-carboxylic Acid Amide

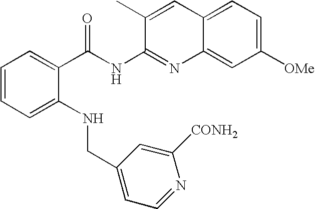

### EXAMPLE 3.2

4-12-(7-Methoxy-2-oxo-2H-chromen-3-ylcarbamoyl)-phenylamino]-methyl}-pyridine-2-carboxylic Acid Amide

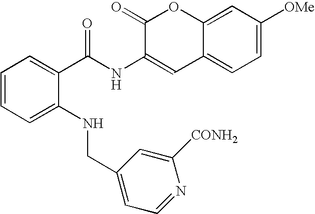

### EXAMPLE 3.3

5-{[2-(7-Methoxy-2-oxo-2H-chromen-3-ylcarbamoyl)-phenylamino]-methyl}-pyridine-2-carboxylic Acid Amide

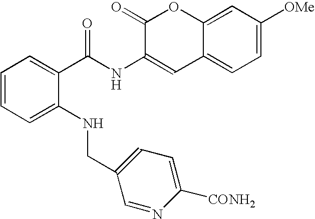

### EXAMPLE 3.4

5-{[2-(7-Methoxy-3-methyl-quinolin-2-ylcarbamoyl)-phenylamino]-methyl}-pyridine-2-carboxylic Acid Amide

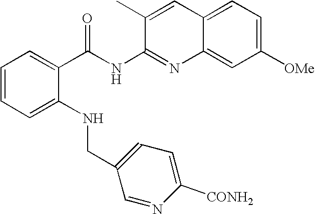

### EXAMPLE 3.5

4-{[2-(Isoquinolin-3-ylcarbamoyl)-6-azaphenylamino]-methyl}-pyridine-2-carboxytic Acid Amide

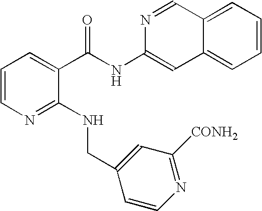

### EXAMPLE 3.6

5-{[2-(2-Oxo-2,3-dihydro-1H-indol-6-ylcarbamoyl)-phenylamino]-methyl}-pyridine-2-carboxylic Acid Amide

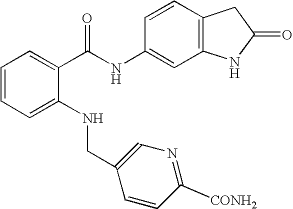

### EXAMPLE 3.7

4-{[2-(2-Methyl-2H-indazol-6-ylcarbamoyl)-phenylamino]-methyl}-pyridine-2-carboxylic Acid Amide

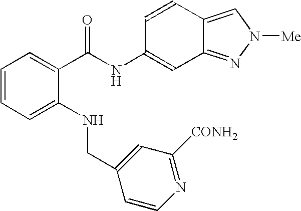

### EXAMPLE 3.8

4-{[2-(1H-Indazol-6-ylcarbamoyl)-phenylamino]-methyl}-pyridine-2-carboxylic Acid Amide

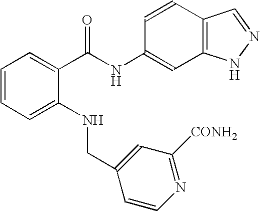

### EXAMPLE 3.9

4-{[2-(1-Methyl-1H-indazol-6-ylcarbamoyl)-phenylamino]-methyl}-pyridine-2-carboxylic Acid Amide

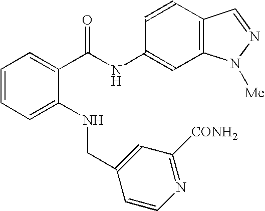

### EXAMPLE 3.10

5-{[2-(Isoquinolin-3-ylcarbamoyl)-6-azaphenylamino]-methyl}-pyridine-2-carboxylic Acid Amide

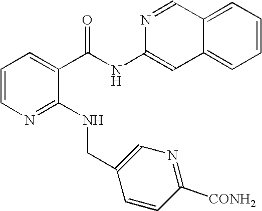

### EXAMPLE 3.11

4-{[2-(2-Oxo-2,3-dihydro-1H-indol-5-ylcarbamoyl)-phenylamino]-methyl}-pyridine-2-carboxylic Acid Amide)

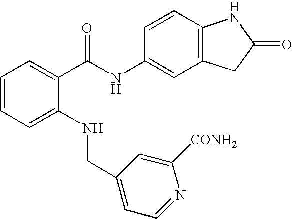

### EXAMPLE 4.0

Production of N-tert-Butyl-(4-{[2-(isoquinolin-3-ylcarbamoyl)-6-azaphenylamino]-methyl}-pyridine-2-carboxylic Acid Amide

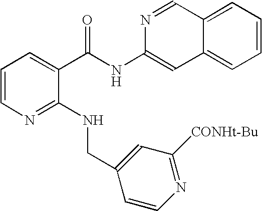

In 5 ml of toluene, 72 mg (0.5 mmol) of 3-aminoisoquinoline is mixed with 0.25 ml of trimethylaluminum (0.5 mmol; 2 mol in toluene) under a cover gas and in a moisture-free environment, and it is stirred for 30 minutes at room temperature. 120 mg (0.45 mmol) of 2-[(2-tert-butylcarbamoyl-pyridin-4-ylmethyl)-amino]-nicotinic acid methyl ester is then added, and it is heated for 2 hours to 120° C. After cooling, it is mixed with 30 ml of dilute sodium bicarbonate solution and extracted three times with 30 ml of ethyl acetate each. The collected ethyl acetate phase is washed with water, dried, filtered and concentrated by evaporation. The residue is chromatographed on silica gel with methylene chloride:ethanol=95:5 as an eluant. After a second chromatography on silica gel with hexane:ethyl acetate=1:1 as an eluant, 70 mg (30% of theory) of N-tert-butyl-(4-{[2-(isoquinolin-3-ylcarbamoyl)-6-azaphenylamino]-methyl}-pyridine-2-carboxylic acid amide) with a melting point of 20¹° C. is obtained.

### EXAMPLE 5.0

Production of 4-{[2-(Isoquinolin-3-ylcarbamoyl)-6-azaphenylamino]-methyl}-pyridine-2-carboxylic Acid (2-Hydroxy-propyl)-amide

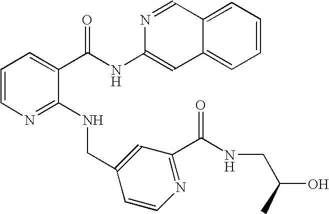

283 mg (1 mmol) of 2-chloro-N-isoquinolin-3-yl-nicotinamide is mixed in 5 ml of pyridine with about 1.66 mmol of 4-aminomethyl-pyridine-2-carboxylic acid (2-hydroxy-propyl)-amide, and it is heated for 2 hours to 100° C. After concentration by evaporation, it is taken up in water and shaken out three times with 30 ml of ethyl acetate each. The collected organic phase is washed with water, dried, filtered and concentrated by evaporation. The residue is chromatographed on silica gel with methylene chloride:acetone 1:1 as an eluant. 40 mg (9% of theory) of 4-{[2-(isoquinolin-3-ylcarbamoyl)-6-azaphenylamino]-methyl}-pyridine-2-carboxylic acid (2-hydroxy-propyl)-amide is obtained as a resin.

### EXAMPLE 6.0

Production of N-(Isoquinolin-3-yl)-2-[3-(pyridin-3-yl)-pyridin-4-yl-methylamino]-benzoic Acid Amide

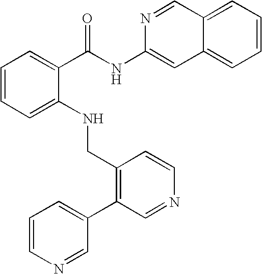

94 mg (0.22 mmol) of N-(isoquinolin-3-yl)-2-[3-bromopyridin-4-yl-methylamino]-benzoic acid amide is mixed in 3.7 ml of toluene in succession with 0.73 ml of ethanol, 0.36 ml of a 2 mol sodium carbonate solution, 6 mg of palladium(0)tetrakistriphenylphosphine and 32 mg of pyridine-3-boronic acid, and it is heated for 6.5 hours to a bath temperature of 120° C. It is then diluted with water to 25 ml and extracted three times with 25 ml of ethyl acetate each. The collected ethyl acetate phase is dried, filtered and concentrated by evaporation. The residue is chromatographed on silica gel with methylene chloride, ethanol=10:1 as an eluant. 45 mg (47% of theory) of N-(isoquinolin-3-yl)-2-[3-(pyridin-3-yl)-pyridin-4-yl-methylamino]-benzoic acid amide is obtained as a resin.

¹H-NMR (d6-DMSO): 10.68 (s, 1H), 9.21 (s, 1H), 8.72 (s, 1H), 8.64 (d, J=3.8, 1H), 8.57 (d, J=5.1, 1H), 8.54 (s, 1H), 8.48 (s, 1H), 8.11-7.94 (m, 4H), 7.85 (d, J=7.6, 1H), 7.74 (t, J=7.3, 1H), 7.59-7.47 (m, 3H), 7.23 (t, J=7.5, 1H), 6.63 (t, J=7.5, 1H), 6.39 (d, J=8.3, 1H), 4.45 (d, J=5.0, 2H).

MS (CI—NH3): 432 (80%, [M+H]⁺)

### EXAMPLE 6.1

Production of N-(Isoquinolin-3-yl)-2-[3-(thien-3-yl)-pyridin-4-yl-methylamino]-benzoic Acid Amide

Similarly produced is also N-(isoquinolin-3-yl)-2-[3-(thien-3-yl)-pyridin-4-yl-methylamino]-benzoic acid amide:

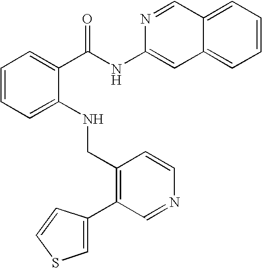

MS (CI—NH3): 437 (100%, [M+H]⁺)

### EXAMPLE 6.2

Production of N-(Isoquinolin-3-yl)-2-[2-amino-carbonylpyridin-4-yl-methylamino]-benzoic Acid Amide

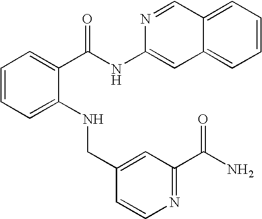

130 mg (0.34 mmol) of N-(isoquinolin-3-yl)-2-[2-cyanopyridin-4-yl-methylamino]-benzoic acid amide is mixed in 2.5 ml of dimethyl sulfoxide with 126 mg of potassium carbonate and 0.25 ml of hydrogen peroxide (30%), and it is stirred for 1 hour at room temperature. It is then mixed with water, and the precipitated product is suctioned off. The residue is absorptively precipitated in a mixture that consists of methylene chloride/ethanol and suctioned off. 96 mg (71% of theory) of N-(isoquinolin-3-yl)-2-[(2-aminocarbonylpyridin-4-yl)-methylamino]-benzoic acid amide with a melting point of 200° C. is obtained.

¹H-NMR (d6-DMSO): 10.73 (s, 1H), 9.22 (s, 1H), 8.60 (s, 1H), 8.56 (d, J=4.7, 1H), 8.25 (br.s, 1H), 8.10-8.04 (m, 3H), 7.95 (d, J=8.0, 1H), 7.88 (d, J=6.9, 1H), 7.74 (t, J=7.4, 1H), 7.63-7.57 (m, 3H), 7.25 (t, J=7.0, 1H), 6.64 (t, J=7.5, 1H), 6.54 (d, J=8.4, 1H), 4.62 (br.d, J=5.5, 2H).

MS (EI): 397 (38%, [M]⁺)

### EXAMPLE 6.3

Production of N-(Isoquinolin-3-yl)-2-[(2-aminocarbonylpyridin-5-yl)-methylamino]-benzoic Acid Amide

Similarly produced is also N-(isoquinolin-3-yl)-2-[(2-aminocarbonylpyridin-5-yl)-methylamino]-benzoic acid amide

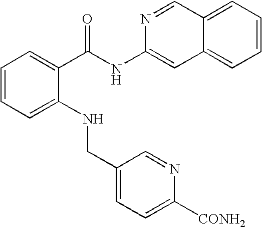

MS (ESI): 398 (78%, [M+H]⁺)

### EXAMPLE 6.4

Production of N-(Isoquinolin-3-yl)-2-[(2-methoxycarbonylpyridin-4-yl)-methylamino]-benzoic Acid Amide

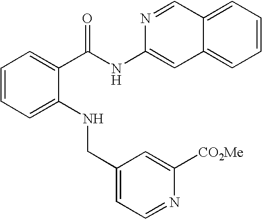

20 mg of N-(isoquinolin-3-yl)-2-[2-bromopyridin-4-yl-methylamino]-benzoic acid amide (0.05 mmol), 1.6 mg (0.003 mmol) of bis(diphenylphosphine)ferrocene (DPPF), 0.35 mg (0.0015 mmol) of palladium(II) acetate, and 14 μl (0.1 mmol) of triethylamine are suspended in a mixture that consists of 1 ml of methanol and 1 ml of dimethylformamide, and it is stirred for 5 hours in an autoclave under CO atmosphere (3 bar) at 50° C. The reaction mixture is filtered using a membrane filter, concentrated by evaporation and chromatographed on silica gel with hexane: EtOAc=3:7 as an eluant. 12 mg (58% of theory) of N-(isoquinolin-3-yl)-2-[(2-methoxycarbonylpyridin-4-yl)-methylamino]-benzoic acid amide is obtained.

¹H-NMR (CDCl₃): 9.12 (br.s, 1H), 8.94 (s, 1H), 8.67 (s, 1H), 8.61 (d, J=5.1, 1H), 8.36 (br.s, 1H), 8.09 (s, 1H), 7.87 (d, J=8.5, 1H), 7.81 (d, J=8.5, 1H), 7.71 (d, J=7.7, 1H), 7.64 (t, J=7.8, 1H), 7.49-7.44 (m, 2H), 7.24-7.19 (m, 1H), 6.68 (t, J=7.8, 1H), 6.42 (d, J=8.0, 1H), 4.50 (br.s, 2H), 3.93 (s, 3H).

MS (ESI): 413 (100%, [M+H]⁺)

### EXAMPLE 6.5

Production of N-(Isoquinolin-3-yl)-2-[(2-benzyloxycarbonylpyridin-4-yl)-methylamino]-benzoic Acid Amide

Similarly produced is also N-(isoquinolin-3-yl)-2-[(2-benzyloxycarbonylpyridin-4-yl)-methylamino]-benzoic acid amide.

¹H-NMR (CDCl₃): 9.00 (s, 1H), 8.76 (br.s, 1H), 8.68 (d, J=5.0, 1H), 8.66 (s, 1H), 8.39 (t, J=6.1, 1H), 8.14 (s, 1H), 7.91 (d, J=7.9, 1H), 7.85 (d, J=8.0, 11H), 7.69-7.62 (m, 2H), 7.53-7.46 (m, 4H), 7.38-7.25 (m, 4H), 6.73 (t, J=7.2, 1H), 6.48 (d, J=7.8, 1H), 5.44 (s, 2H), 4.56 (d, J=6.0, 2H).

MS (CI—NH3): 489 (85%, [M+H]⁺)

### EXAMPLE 6.6

Production of N-(Isoquinolin-3-yl)-2-[(2-hydroxycarbonylpyridin-4-yl)-methylamino]-benzoic Acid Amide

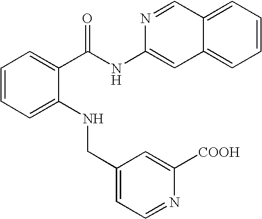

a. 20 mg of N-(isoquinolin-3-yl)-2-[(2-methoxycarbonylpyridin-4-yl)-methylamino]-benzoic acid amide (0.05 mmol) is mixed in a mixture that consists of 1 ml of tetrahydrofuran and 1 ml of methanol with 10.2 mg (0.25 mmol) of lithium hydroxide in water, and it is stirred for 4 hours at 22° C. The reaction mixture is filtered using a membrane filter, concentrated by evaporation and chromatographed on silica gel with toluene:acetic acid:water 10:10:1 as an eluant. 14 mg (69% of theory) of N-(isoquinolin-3-yl)-2-[(2-hydroxy-carbonylpyridin-4-yl)-methylamino]-benzoic acid amide is obtained.

b. 433 mg of N-(isoquinolin-3-yl)-2-[2-bromopyridin-4-yl)-methylamino]-benzoic acid amide (1 mmol), 50 mg (0.09 mmol) of bis(diphenylphosphine)ferrocene (DPPF), 10 mg (0.045 mmol) of palladium(II) acetate, and 280 μl (2 mmol) of triethylamine are suspended in a mixture that consists of 5 ml of water and 10 ml of dimethylformamide, and it is stirred for 5 hours in an autoclave under CO atmosphere (3 bar) at 50° C. The reaction mixture is filtered using a membrane filter, concentrated by evaporation, dissolved in dichloromethane, mixed with activated carbon, heated, filtered and concentrated by evaporation. The solid that is obtained is recrystallized from dichloromethane. 283 mg (71% of theory) of N-(isoquinolin-3-yl)-2-[(2-hydroxycarbonylpyridin-4-yl)-methylamino]-benzoic acid amide is obtained.

¹H-NMR (d6-DMSO): 10.73 (s, 1H), 9.22 (s, 1H), 8.63 (d, J=4.9, 1H), 8.60 (s, 1H), 8.22 (br.t, J=6.0, 1H), 8.10 (d, J=8.0, 1H), 8.04 (s, 1H), 7.94 (d, J=8.1, 1H), 7.87 (d, J=6.8, 1H), 7.74 (t, J=7.5, 1H), 7.60-7.54 (m, 2H), 7.25 (t, J=7.0, 1H), 6.65 (t, J=7.6, 1H), 6.54 (d, J=8.4, 1H), 4.62 (br.d, J=5.5, 2H). A proton is not observed or is masked.

MS(CI—NH3): 399 (75%, [M+H]⁺)

Melting point: 185° C.

### EXAMPLE 6.7

Production of N-(isoquinolin-3-yl)-2-[(2-morpholinocarbonylpyridin-4-yl)-methylamino]-benzoic Acid Amide

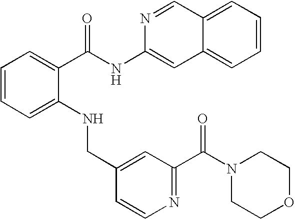

A mixture that consists of 40 mg of N-(isoquinolin-3-yl)-2-[(2-hydroxycarbonylpyridin-4-yl)-methylamino]-benzoic acid amide (0.1 mmol) and 9 μl (0.1 mmol) of morpholine in 1 ml of dimethylformamide is mixed in portions with 34 mg (0.2 mmol) of carbonyldiimidazole. After 4 hours of stirring at 22° C., it is concentrated by evaporation, the residue is dissolved in 5 ml of dichloromethane, washed with 1 mol of aqueous potassium carbonate solution (2 ml), dried (MgSO₄), filtered and concentrated by evaporation. Colorless resin (38 mg, 81% of theory).

¹H-NMR (CDCl₃): 9.02 (s, 1H), 8.71 (br.s, 1H), 8.64 (s, 1H), 8.51 (d, J=5.1, 1H), 8.36 (t, J=6.0, 1H), 8.01 (s, 1H), 7.93-7.82 (m, 2H), 7.69-7.64 (m, 2H), 7.50 (t, J=7.8, 1H), 7.39 (d, J=6.1, 1H), 7.28-7.20 (m, 1H), 6.73 (t, J=7.8, 1H), 6.52 (d, J=8.1, 1H), 4.55 (d, J=6.0, 2H), 3.79-3.62 (m, 8H).

MS (EI): 467 (15%, [M+H]⁺)

### EXAMPLE 6.8

Production of N-(Isoquinolin-3-yl)-2-[3-trimethylsilylethinylpyridin-4-yl)-methylamino]-benzoic Acid Amide

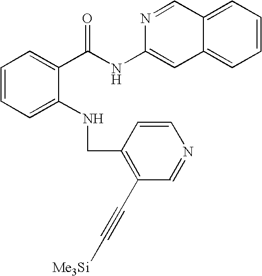

108 mg (0.25 mmol) of N-(isoquinolin-3-yl)-2-[3-bromopyridin-4-yl)-methylamino]-benzoic acid amide is mixed in 1 ml of dimethylformamide with 1 ml of triethylamine, 5 mg (0.026 mmol) of copper-1-iodide, 9 mg (0.008 mmol) of palladium tetrakis triphenylphosphine and 0.07 ml of trimethylsilylacetylene, and it is heated under argon and in a moisture-free environment for 3.5 hours to a bath temperature of 70° C. It is then mixed with 40 ml of water and extracted three times with 25 ml of ethyl acetate each. The ethyl acetate phase is washed with water, dried, filtered and concentrated by evaporation. The residue is chromatographed on silica gel with ethyl acetate:hexane=1:1 as an eluant. 38 mg (33.6% of theory) of N-(isoquinolin-3-yl)-2-[3-trimethylsilyl-ethinylpyridin-4-yl)-methylamino]-benzoic acid amide is obtained as an amorphous solid.

¹H-NMR (d6-DMSO): 10.71 (s, 1H), 9.22 (s, 1H), 8.62 (s, 1H), 8.58 (s, 1H), 8.49 (d, J=4.9, 1H), 8.21 (br.t, J=6.1, 1H), 8.09 (d, J=8.2, 1H), 7.92 (d, J=8.0, 1H), 7.88 (d, J=7.9, 1H), 7.74 (t, J=8.0, 1H), 7.57 (t, J=7.7, 1H), 7.40 (d, J=5.1, 1H), 7.28 (t, J=7.5, 1H), 6.65 (t, J=7.7, 1H), 6.54 (d, J=8.1, 1H), 4.58 (d, J=6.0, 2H), 0.27 (s, 3H).

MS (EI): 450 (105%, [M]⁺)

### EXAMPLE 6.9

Production of N-(Isoquinolin-3-yl)-2-[2-trimethylsilylethinylpyridin-4-yl)-methylamino]-benzoic Acid Amide

Produced in a way similar to Example 9 is also N-(isoquinolin-3-yl)-2-[2-trimethylsilylethinylpyridin-4-yl)-methylamino]-benzoic acid amide

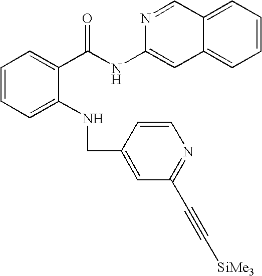

Production of Starting and Intermediate Compounds

If the production of the intermediate compounds is not described, the latter are known or can be produced analogously to known compounds or processes that are described here.

### EXAMPLE A

Process Stage 1

A-1) Production of 2-Bromopyridine-5-carbaldehyde

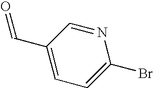

2-Bromopyridine-5-carbaldehyde is produced according to F. J. Romero-Salguerra et al. THL 40, 859 (1999).

A-2) Production of 2-Bromo-isonicotinic Acid

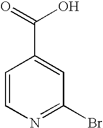

160 g (0.93 mol) of 2-bromo-4-methyl-pyridine is added in drops to 152 g (0.96 mol) of potassium permanganate in 4 l of water. Then, it is stirred under reflux for one hour before 152 g (0.96 mol) of potassium permanganate is added once again. After two additional hours of stirring under reflux, it is suctioned off in a hot state over Celite and washed with water. The aqueous phase is shaken out three times with dichloromethane. The aqueous phase is concentrated by evaporation to one-half of its original volume and set at pH 2 with concentrated hydrochloric acid. The precipitated solid is suctioned off and dried at 70° C. in a vacuum. 56.5 g (28% of theory) of 2-bromo-isonicotinic acid accumulates as a white solid product.

A-3) Production of 2-Bromo-4-hydroxymethyl-pyridine

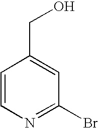

30.2 ml (295 mmol) of triethylamine is added to 56.5 g (280 mmol) of 2-bromo-isonicotinic acid in 1.2 l of tetrahydrofuran (THF). Then, it is cooled to −10° C. and mixed drop by drop with 38.2 ml (295 mmol) of isobutyl formate. After one hour at −10° C., stirring was continued, it is cooled to −70° C. and mixed drop by drop with 590 ml (590 mmol) of lithium aluminum hydride (LiAlH₄) solution (1 M in THF). After stirring is continued for one hour at −70° C., it is allowed to reach −40° C. 600 ml of 50% acetic acid is added. It is stirred overnight at room temperature. The insoluble components are suctioned off, and the filtrate is concentrated by evaporation. The residue is purified on silica gel with hexane and hexane/ethyl acetate 1:1. 28.0 g (55% of theory) of 2-bromo-4-hydroxymethyl-pyridine accumulates as a white solidifying oil.

A4) Production of 2-Bromo-4-formyl-pyridine

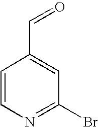

149 g (1714 mmol) of manganese dioxide is added in measured quantities in 6 hours to 28.0 g (148.9 mmol) of 2-bromo-4-hydroxymethyl-pyridine in 500 ml of dichloromethane. Then, it is stirred for 48 more hours at room temperature. It is suctioned off on Celite and concentrated by evaporation. 16.4 g (60% of theory) of 2-bromo-4-formyl-pyridine accumulates as a solidifying white oil.

Process Stage 2

A-5) Production of 2-[(6-Bromo-pyridin-3-ylmethyl)-amino]-N-isoquinolin-3-yl-benzamide

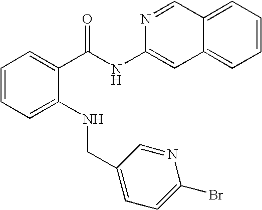

3.46 g (13.17 mmol) of 2-amino-N-isoquinolin-3-yl-benzamide is introduced into 50 ml of methanol, mixed with 1.5 ml of glacial acetic acid as well as 2.45 g (13.17 mmol) of 2-bromopyridine-5-carbaldehyde and stirred for 24 hours under argon and in a moisture-free environment at room temperature. Then, it is mixed with 828 mg (13.17 mmol) of sodium cyanoborohydride and stirred for another 24 hours at room temperature. After concentration by evaporation under a vacuum, the residue is taken up in dilute sodium bicarbonate solution and suctioned off. The residue that is obtained is absorptively precipitated in a little ethyl acetate and suctioned off again. The residue that is obtained in this case is on silica gel with hexane:ethyl acetate=1:1 as an eluant. 3.27 g (57% of theory) of 2-[(6-bromo-pyridin-3-ylmethyl)-amino]-N-isoquinolin-3-yl-benzamide is obtained.

A-6) Production of N-(Isoquinolin-3-yl)-2-[3-bromopyridin-4-yl-methylamino]-benzoic Acid Amide

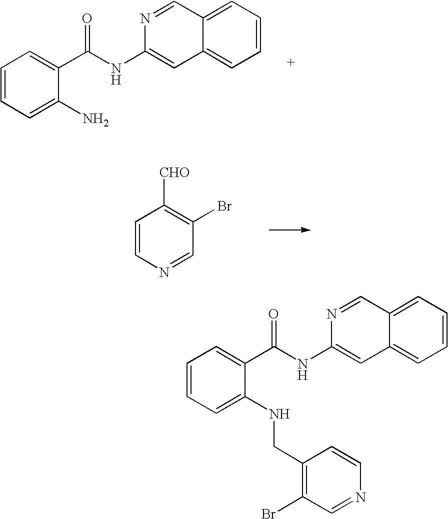

263 mg (1 mmol) of N-(isoquinolin-3-yl)-2-aminobenzoic acid amide is mixed in 6 ml of MeOH in succession with 0.06 ml of glacial acetic acid, 298 mg (1.6 mmol) of 3-bromo-pyridine-4-carbaldehyde (produced according to Tetrahedron 2000, 347), and it is stirred for 24 hours at room temperature. Then, 100 mg (1.6 mmol) of sodium cyanoborohydride is added, and it is stirred for another 24 hours. It is then mixed with 50 ml of dilute sodium bicarbonate solution, and the precipitated product is suctioned off. The residue is chromatographed on silica gel with methylene chloride:ethanol=95:5 as an eluant. N-(Isoquinolin-3-yl)-2-[3-bromopyridin-4-yl-methylamino]-benzoic acid amide is obtained as a resin. The 3-bromo-pyridine-4-carbaldehyde that is used is produced according to Chem. Pharm. Bull. 1970, 38, 2446.

Similarly produced is:

A-7) 2-[(2-Bromo-pyridin-4-ylmethyl)-amino]-N-isoquinolin-3-yl-benzamide

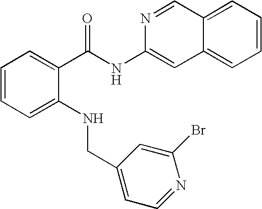

¹H-NMR (CDCl₃): 9.00 (s, 1H), 8.78 (s, 1H), 8.66 (s, 1H), 8.35 (t, J=5.7, 1H), 8.30 (d, J=5.1, 1H), 7.92 (d, J=8.1, 1H), 7.86 (d, J=8.5, 1H), 7.70-7.65 (m, 2H), 7.53-7.48 (m, 2H), 7.33-7.26 (m, 2H), 6.75 (t, J=7.8, 1H), 6.48 (d, J=8.5, 1H), 4.48 (d, J=5.9, 2H).

MS (CI, NH3): 435 (100%), 433 (100%)

A-8) 2-[(2-Bromo-pyridin-4-ylmethyl)-amino]-N-(2-oxo-2,3-dihydro-2H-indol-6-yl)-Benzamide

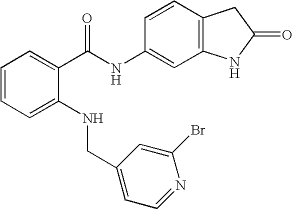

A-9) 2-[(2-Bromo-pyridin-4-ylmethyl)-amino]-N-(2-oxo-2,3-dihydro-1H-indol-5-yl)-benzamide

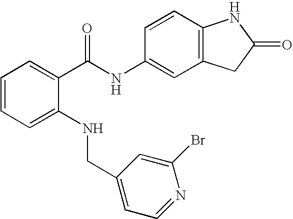

A-10) 2-[(6-Bromo-pyridin-3-ylmethyl)-amino]-N-(2-oxo-2,3-dihydro-1H-indol-6-yl)-Benzamide

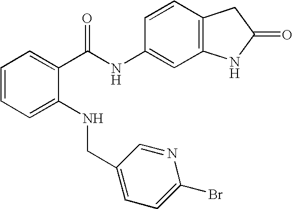

A-11) 2-[(6-Bromo-pyridin-3-ylmethyl)-amino]-N-(2-oxo-2,3-dihydro-1H-indol-5-yl)-benzamide

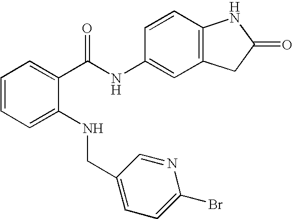

A-12) 2-[(6-Bromo-pyridin-3-ylmethyl)-amino]-N-(7-methoxy-2-oxo-2H-chromen-3-yl)-benzamide

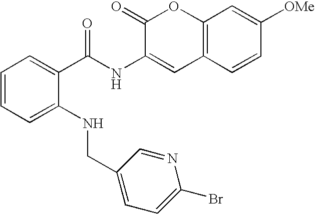

A-13) 2-[(2-Bromo-pyridin-4-ylmethyl)-amino]-N-(7-methoxy-2-oxo-2H-chromen-3-yl)-benzamide

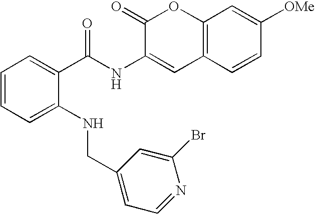

A-14a) 2-[(2-Bromo-pyridin-4-ylmethyl)-amino]-N-(3-trifluoromethyl-phenyl)-Benzamide

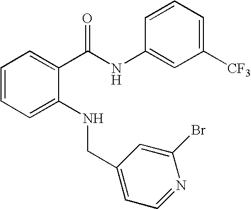

A-14b) 2-[(6-Bromo-pyridin-3-ylmethyl)-amino]-N-(7-methoxy-3-methylquinolin-2-yl)-benzamide

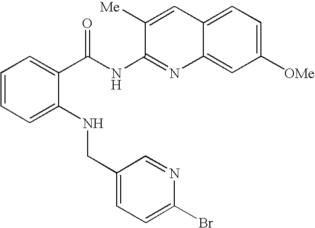

Process Stage 3

A-15) Production of 5-{[2-(Isoquinolin-3-ylcarbamoyl)-phenylamino]-methyl}-pyridine-2-carboxylic Acid

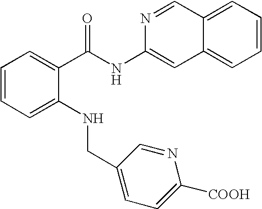

3.27 g (7.55 mmol) of 2-[(6-bromo-pyridin-3-ylmethyl)-amino]-N-isoquinolin-3-yl-benzamide is mixed in 75 ml of dimethylformamide with 2.2 ml of triethylamine, 36 ml of water, 362 mg (0.65 mmol) of bisdiphenylphosphinoferrocene and 75 mg (0.33 mmol) of palladium(II) acetate, and it is shaken in an autoclave under carbon monoxide at a pressure of 3 bar and a temperature of 50° C. for 3 hours. After cooling, it is suctioned off on diatomaceous earth and concentrated by evaporation. The residue is taken up in water, set at pH 5-6 with glacial acetic acid, suctioned off, and the filter cakes are rewashed with hexane. 3.35 g of 5-{[2-(isoquinolin-3-ylcarbamoyl)-phenylamino]-methyl}-pyridine-2-carboxylic acid, which is further reacted without further purification, is obtained.

Similarly produced are:

A-16) 4-{[2-(Isoquinolin-3-ylcarbamoyl)-phenylamino]-methyl}-pyridine-2-carboxylic Acid

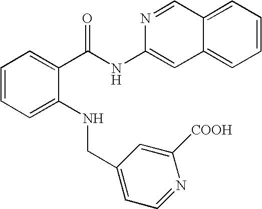

A-17) 4-{[2-(2-Oxo-2,3-dihydro-1H-indol-6-ylcarbamoyl)-phenylamino]-methyl}-pyridine-2-carboxylic Acid

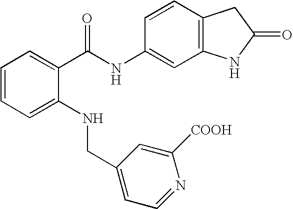

A-18) 5-{[2-(2-Oxo-2,3-dihydro-1H-indol-6-ylcarbamoyl)-phenylamino]-methyl}-pyridine-2-carboxylic Acid

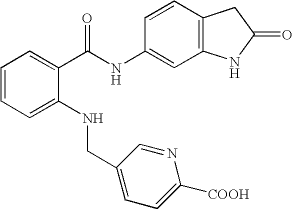

A-19) 4-{[2-(2-Oxo-2,3-dihydro-1H-indol-5-ylcarbamoyl)-phenylamino]-methyl}-pyridine-2-carboxylic Acid

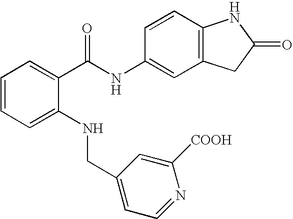

A-20) 5-{[2-(2-Oxo-2,3-dihydro-1H-indol-5-ylcarbamoyl)-phenylamino]-methyl}-pyridine-2-carboxylic Acid

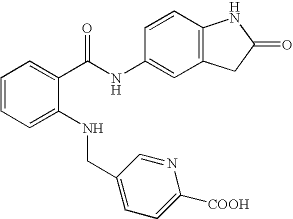

A-21) 5-{[2-(7-Methoxy-2-oxo-2H-chromen-3-ylcarbamoyl)-phenylamino]-methyl}-pyridine-2-carboxylic Acid

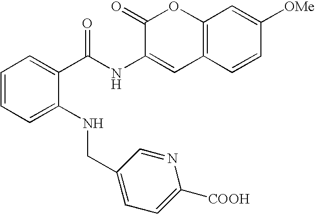

A-22) 4-{[2-(7-Methoxy-2-oxo-2H-chromen-3-ylcarbamoyl)-phenylamino]-methyl}-pyridine-2-carboxylic Acid

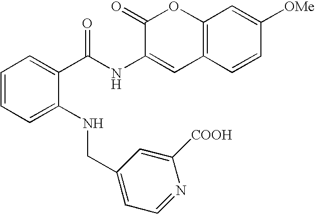

A-23) 4-{[2-(7-Methoxy-3-methyl-quinolin-2-ylcarbamoyl)-phenylamino]-methyl}-pyridine-2-carboxylic Acid

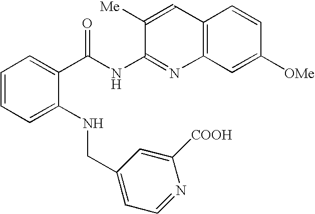

A-24) 5-{[2-(7-Methoxy-3-methyl-quinolin-2-ylcarbamoyl)-phenylamino]-methyl}-pyridine-2-carboxylic Acid

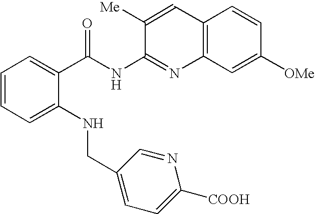

A-25) 5-{[2-(1-Methyl-1H-indazol-6-ylcarbamoyl)-phenylamino]-methyl}-pyridine-2-carboxylic Acid

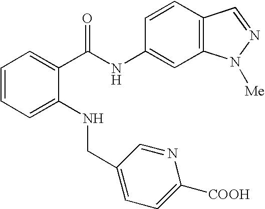

A-26) 4-{[2-(1-Methyl-1H-indazol-6-ylcarbamoyl)-phenylamino]-methyl}-pyridine-2-carboxylic Acid

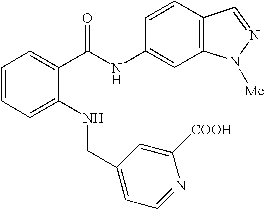

A-27) 4-{[2-(2-Methyl-2H-indazol-6-ylcarbamoyl)-phenylamino]-methyl}-pyridine-2-carboxylic Acid

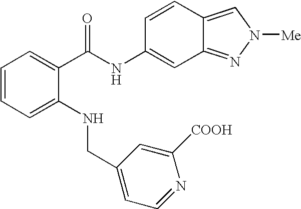

A-28) 5-{[2-(2-Methyl-2H-indazol-6-ylcarbamoyl)-phenylamino]-methyl}-pyridine-2-carboxylic Acid

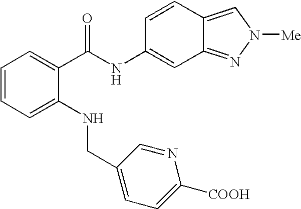

A-29) 4-{[2-(3-Trifluoromethyl-phenylcarbamoyl)-phenylamino]-methyl}-pyridine-2-carboxylic Acid

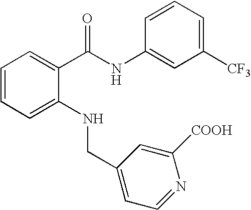

Melting point 151° C.

A-30) 4-{[2-(1H-Indazol-6-ylcarbamoyl)-phenylamino]methyl}-pyridine-2-carboxylic Acid

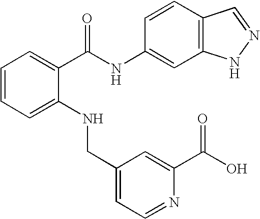

A-31) 4-{[2-(1H-Indazol-5-ylcarbamoyl)-phenylamino]methyl}-pyridine-2-carboxylic Acid

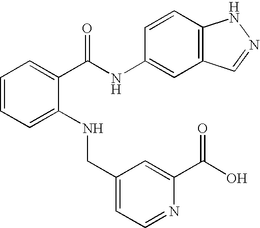

### EXAMPLE B

Process Stage 1

B-1) Production of 5-Nitro-1,3-dihydro-indol-2-one

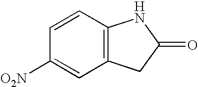

5-Nitro-1,3-dihydro-indol-2-one is produced according to R. T. Courts, J. Org. Chem. 48, 3747, (1970).

B-2) Production of Dinitrophenylacetic Acid Methyl Ester.

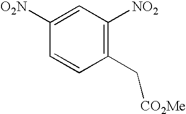

22.6 g (100 mmol) of 2,4-dinitrophenylacetic acid is dissolved in a mixture of 200 ml of methanol and 830 ml of toluene and mixed at room temperature with 83 ml of trimethylsilyldiazomethane (2 mol in toluene; 166 mmol), and it is stirred for 3 hours at room temperature. After evaporation to the dry state and drying at 70° C. in a vacuum, 24 g (100% of theory) of 2,4-dinitrophenylacetic acid methyl ester is obtained.

B-3) Production of 6-Nitro-1,3-dihydro-indol-2-one

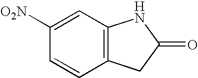

20 g (83 mmol) of 2,4-dinitrophenylacetic acid methyl ester is hydrogenated in 400 ml of glacial acetic acid with 2.1 g of palladium/carbon (10%) under 20 bar of hydrogen for 1.5 hours at room temperature. After catalyst is filtered out, it is concentrated by evaporation and very quickly dried on solid potassium hydroxide in a vacuum. The residue is chromatographed on silica gel with a gradient that consists of methylene chloride:ethanol=97.5:2.5 to 90:10 as an eluant. After recrystallization from ethyl acetate, 4 g (30% of theory) of 6-nitro-1,3-dihydro-indol-2-one with a melting point of 206° C. is obtained.

Process Stage 2

B4) Production of 5-Amino-1,3-dihydro-indol-2-one

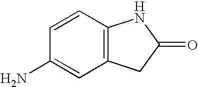

356 mg of 5-nitro-1,3-dihydro-indol-2-one is hydrogenated in 30 ml of tetrahydrofuran:ethanol=1:1 with 400 mg of palladium on carbon (10%) at room temperature and normal pressure for 1 hour. After the catalyst is suctioned off on diatomaceous earth and after concentration by evaporation, 320 mg (100% of theory) of 5-amino-1,3-dihydro-indol-2-one is obtained.

B-5) Production of 6-Amino-1,3-dihydro-indol-2-one

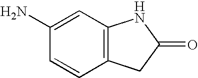

Similarly produced from the corresponding nitro compound is 6-amino-1,3-dihydro-indol-2-one.

Process Stage 3

B-6) 2-Nitro-N-(2-oxo-2,3-dihydro-1H-indol-5-yl)-benzamide

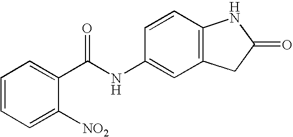

320 mg of 5-amino-1,3-dihydro-indol-2-one is dissolved in 1 ml of dimethylacetamide and mixed drop by drop with 371 mg (2 mmol) of 2-nitrobenzoyl chloride, whereby a slight heating occurs. After stirring overnight at room temperature, it is concentrated by evaporation in a vacuum, and the residue is taken up in ethyl acetate and water. The suctioning-off of an insoluble solid provides 130 mg (21.9% of theory) of 2-nitro-N-(2-oxo-2,3-dihydro-1H-indol-5-yl)-benzamide. After shaking out, the organic phase is washed, filtered and concentrated by evaporation, and 400 mg (67% of theory) of 2-nitro-N-(2-oxo-2,3-dihydro-1H-indol-5-yl)-benzamide with a melting point of 265° C. is obtained again.

B-7) Production of 2-Nitro-N-(2-oxo-2,3-dihydro-1H-indol-6-yl)-benzamide

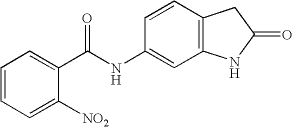

Produced similarly to I) is 2-nitro-N-(2-oxo-2,3-dihydro-1H-indol-6-yl)-benzamide with a melting point >300° C.

Process Stage 4

B-8) Production of 2-Amino-N-(indol-2-on-5-yl)benzoic Acid Amide

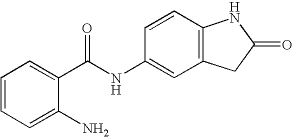

Produced in a way similar to process stage 2 is also 2-amino-N-(indol-2-on-5-yl)benzoic acid amide with a melting point of 219° C.

B-9) Production of 2-Amino-N-(indol-2-on-6-yl)benzoic Acid Amide

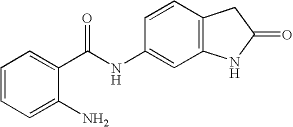

Produced in a way similar to stage 2 is also 2-amino-N-(indol-2-on-6-yl)benzoic acid amide with a melting point of 230° C.

### EXAMPLE C

C-1) Production of 2-Amino-N-(7-methoxy-2-oxo-2H-chromen-3-yl)-benzamide

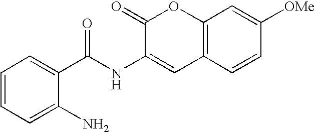

Process Stage 1

C-2) Production of 3-Nitro-7-methoxy-chromen-2-one

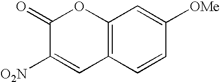

13 g (85.4 mmol) of 2-hydroxy-4-methoxybenzaldehyde is heated in 300 ml of toluene with 9.8 g (102.5 mmol) of n-propylamine hydrochloride and 11.5 ml (102.5 mmol) of nitroacetic acid ethyl ester for 15 hours in a water separator. 3 ml of nitroacetic acid ethyl ester is then added again and boiled for another 5 hours in a water separator. After cooling, it is diluted with ethyl acetate and shaken out with water. The ethyl acetate phase is dried, filtered and concentrated by evaporation. The residue is chromatographed on silica gel with methylene chloride as an eluant. 6.14 g (33% of theory) of 3-nitro-7-methoxy-chromen-2-one is obtained.

Process Stage 2

C-3) Production of 3-Amino-7-methoxy-chromen-2-one

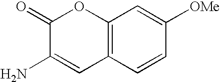

In a way similar to process stage 2 of Example B, 3-amino-7-methoxy-chromen-2-one is produced from 3-nitro-7-methoxy-chromen-2-one in ethanol.

Process Stage 3

C4) Production of 2-Nitro-N-(7-methoxybenzopyran-2-on-3-yl)benzoic Acid Amide

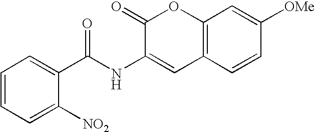

In a way similar to process stage 3 from Example B, 2-nitro-N-(7-methoxybenzopyran-2-on-3-yl)benzoic acid amide is produced from 2-nitrobenzoyl chloride and 3-amino-7-methoxy-chromen-2-one and 2-nitro-N-(7-methoxy-2-oxo-2H-chromen-3-yl)-benzamide.

Process Stage 4

C-5) Production of 2-Amino-N-(7-methoxy-2-oxo-2H-chromen-3-yl)-Benzamide

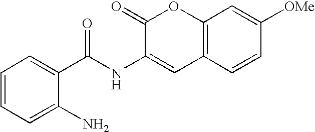

In a way similar to process stage 2 from Example B, the 2-amino-N-(7-methoxy-2-oxo-2H-chromen-3-yl)-benzamide is produced from 2-nitro-N-(7-methoxy-2-oxo-2H-chromen-3-yl)-benzamine in ethanol:tetrahydrofuran=5:2.

### EXAMPLE D

D-1) Production of 2-[(6-Cyano-pyridin-3-ylmethyl)-amino]-N-(2-oxo-2,3-dihydro-1H-indol-5-yl)-benzamide

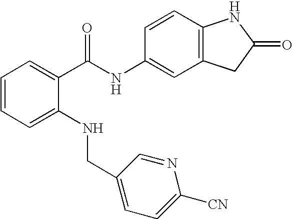

219 mg (0.5 mmol) of 2-[(6-bromo-pyridin-3-ylmethyl)-amino]-N-(2-oxo-2,3-dihydro-1H-indol-5-yl)-benzamide is added into 7 ml of dimethyl acetamide with 59 mg (0.5 mmol) of zinc(II)cyanide, 12 mg (0.013 mmol) of tris(dibenzylidene acetone)-dipalladium, 10 mg (0.018 mmol) of bis(diphenylphosphino)ferrocene and 4 mg (0.06 mmol) of zinc powder, and it is stirred under argon and in a moisture-free environment for 7.5 hours at a bath temperature of 150° C. After cooling, it is diluted with water, shaken out with ethyl acetate, and the organic phase is dried, filtered and concentrated by evaporation. The residue is chromatographed on silica gel with a gradient of methylene chloride:ethanol=97.5:2.5 to 90:10 as an eluant. 65 mg (30% of theory) of2-[(6-cyano-pyridin-3-ylmethyl)-amino]-N-(2-oxo-2,3-dihydro-1H-indol-5-yl)-benzamide is obtained.

Similarly produced are:

D-2) 2-[(6-Cyano-pyridin-3-ylmethyl)-amino]-N-(7-methoxy-2-oxo-2H-chromen-3-yl)-benzamide

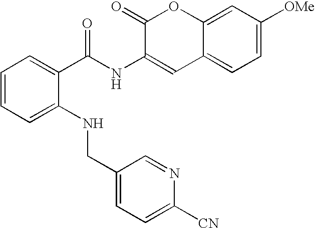

### EXAMPLE E

Process Stage 1

E-1) Production of 2-Chloro-nicotinic Acid Methyl Ester

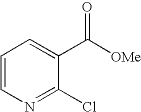

5.6 g of 2-chloro-nicotinic acid is dissolved in 280 ml of toluene and 80 ml of methanol and mixed with 37.4 ml (74.8 mmol) of trimethylsilyldiazomethane (2 mol in hexane) and stirred for 3 hours at room temperature. After the batch is concentrated by evaporation, 7 g (100% of theory) of 2-chloro-nicotinic acid methyl ester is obtained.

Process Stage 2

E-2) Production of 2-[(Pyridin-4-ylmethyl)-amino]-nicotinic Acid Methyl Ester

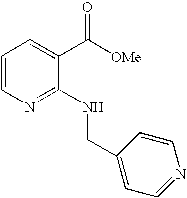

4.0 g (23.3 mmol) of 2-chloro-nicotinic acid methyl ester is heated with 2.52 g (23.3 mmol) of 4-aminomethylpyridine for 1.5 hours to a bath temperature of 100° C. After cooling, it is diluted with 100 ml of dilute sodium bicarbonate solution and shaken out three times with 50 ml of ethyl acetate each. The combined organic phase is washed, dried, filtered and concentrated by evaporation. The residue is chromatographed on silica gel with methylene chloride:ethanol=10:1 as an eluant. 1.36 g (24% of theory) of 2-[(pyridin-4-ylmethyl)-amino]-nicotinic acid methyl ester is obtained.

Similarly produced is:

E-3) 2-[(Pyridin-3-ylmethyl)-amino]-nicotinic Acid Methyl Ester

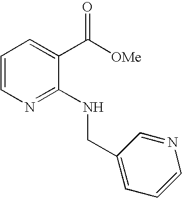

Process Stage 3

E4) Production of 2-[(1-Oxy-pyridin-4-ylmethyl)-amino]-nicotinic Acid Methyl Ester

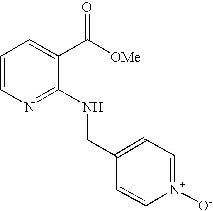

2.09 g (8.59 mmol) of 2-[(pyridin-4-ylmethyl)-amino]-nicotinic acid methyl ester is mixed in 150 ml of methylene chloride with 2.21 g (9.88 mmol) of m-chloroperbenzoic acid, and it is stirred for 24 hours at room temperature. It is mixed with 50 ml of dilute sodium bicarbonate solution, shaken, the organic phase is separated and extracted three times with 50 ml of methylene chloride each. The combined organic phase is washed, dried, filtered and concentrated by evaporation. 2.7 g (100% of theory) of 2-[(1-oxy-pyridin-4-ylmethyl)-amino]-nicotinic acid methyl ester is obtained as an oil.

Similarly produced is:

E-5) 2-[(1-Oxy-pyridin-3-ylmethyl)-amino]-nicotinic Acid Methyl Ester

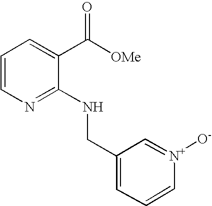

Process Stage 4

E-6) Production of 2-[(2-Cyano-pyridin-4-ylmethyl)-amino]-nicotinic Acid Methyl Ester

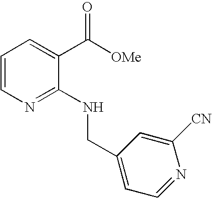

2.7 g (10.4 mmol) of 2-[(1-oxy-pyridin-4-ylmethyl)-amino]-nicotinic acid methyl ester is heated in 52 ml of dimethylformamide in a pressure vessel together with 3.15 g (31.2 mmol) of triethylamine and 9.19 g (62.4 mmol) of trimethylsilyl cyanide for 8 hours to a bath temperature of 110° C. After concentration by evaporation in a vacuum, the residue is taken up in 100 ml of dilute sodium bicarbonate solution and extracted three times with 100 ml of ethyl acetate each. The combined organic phase is washed, dried, filtered and concentrated by evaporation. The residue is chromatographed on a flash column (50 g; Isolute flash silica; Separtis Company) with a gradient of methylene chloride:ethanol=100:0 to 95:5 as an eluant. 1.31 g (47% of theory) of 2-[(2-cyano-pyridin-4-ylmethyl)-amino]-nicotinic acid methyl ester is obtained.

Similarly produced is:

E-7) Production of 2-[(6-Cyano-pyridin-3-ylmethyl)-amino]-nicotinic Acid Methyl Ester

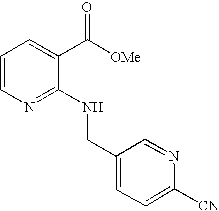

In the production, a small amount of 2-[(2-cyano-pyridin-3-ylmethyl)-amino]-nicotinic acid methyl ester accumulates at the same time.

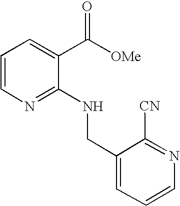

Process Stage 5

E-8) Production of 2-[(2-Cyano-pyridin-4-ylmethyl)-amino]-N-isoquinolin-3-yl-nicotinamide

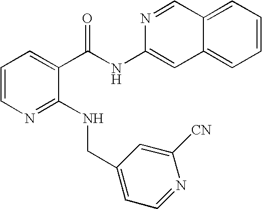

In 10 ml of toluene, 277 mg (1.92 mmol) of 3-aminoisoquinoline and 0.86 ml of trimethylaluminum (2 mol solution in toluene) are stirred under argon and in a moisture-free environment for 30 minutes at 4° C. 468 mg (1.74 mmol) of 2-[(2-cyano-pyridin-4-ylmethyl)-amino]-nicotinic acid methyl ester is then added and refluxed for 2 hours. It is mixed with 30 ml of dilute sodium bicarbonate solution and shaken out three times with 30 ml of ethyl acetate each. The combined organic phase is washed, dried, filtered and concentrated by evaporation. The residue is chromatographed on a flash column (20 g; Isolute flash silica; Separtis Company) with a gradient of methylene chloride:ethanol=100:0 to 95:5 as an eluant. 400 mg (60% of theory) of 2-[(2-cyano-pyridin-4-ylmethyl)-amino]-N-isoquinolin-3-yl-nicotinamide is obtained.

E-9) N-(Isoquinolin-3-yl)-2-[2-cyanopyridin-4-yl-methylamino]-benzoic Acid Amide

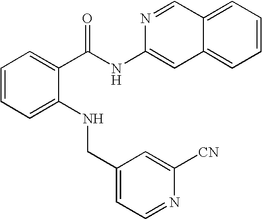

920 mg (2.5 mmol) of N-(isoquinolin-3-yl)-2-(4-pyridylmethyl)-aminobenzoic acid amide-N-oxide is mixed in a glass pressure vessel in succession with 20 ml of dimethylformamide in succession with 760 mg (7.5 mmol) of triethylamine and 1.24 g (12.5 mmol) of trimethylsilylcyanide and then heated for 10 hours to a bath temperature of 110° C. It is then diluted with water to about 200 ml and shaken out three times with 50 ml of ethyl acetate each. The collected organic phase is washed with 50 ml of water, dried, filtered and concentrated by evaporation. The residue is chromatographed first on silica gel with ethyl acetate:hexane=1:1 and then again on silica gel with dichloromethane:ethanol=100:2 as an eluant. 132 mg (14% of theory) of N-(isoquinolin-3-yl)-2-(4-2-cyanopyridylmethyl)amino-benzoic acid amide is obtained as a resin.

If the production of the intermediate compounds is not described, the latter are known or can be produced analogously to the known compounds or the processes that are described here.

Similarly produced are:

E-10) 2-[(2-Cyano-pyridin-3-ylmethyl)-amino]-N-isoquinolin-3-yl-nicotinamide

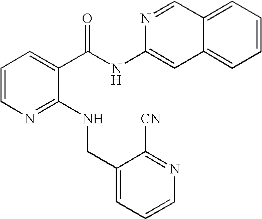

E-11) 2-[(6-Cyano-pyridin-3-ylmethyl)-amino]-N-isoquinotin-3-yl-nicotinamide

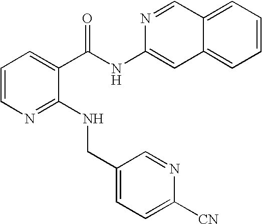

### EXAMPLE F

1. Process Stage

F-1) Production of 2-[(2-Bromo-pyridin-4-ylmethyl)-amino]-benzoic Acid-Methyl Ester

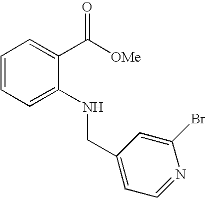

6.04 g (40 mmol) of anthranilic acid methyl ester in 600 ml of methanol is mixed with 3.2 ml of acetic acid and 7.4 g (40 mmol) of 2-bromopyridine-4-carbaldehyde and stirred overnight at 40° C. 3.8 g (60 mmol) of sodium cyanoborohydride is added thereto, and it is stirred overnight at 40° C. 3.8 g (60 mmol) of sodium cyanoborohydride is added again and stirred over the weekend at 40° C. It is mixed with water and largely concentrated by evaporation. The aqueous phase is extracted with ethyl acetate, and the combined organic phases are dried, filtered and concentrated by evaporation. The crude product is chromatographed on silica gel with a gradient that consists of hexane and hexane/ethyl acetate 1:3 and hexane/ethyl acetate 1:1 as an eluant. 10.0 g (78% of theory) of 2-[(2-bromo-pyridin-4-ylmethyl)-amino]-benzoic acid methyl ester is obtained as a colorless oil.

2. Process Stage

F-2) Production of 2-[(2-Cyano-pyridin-4-ylmethyl)-amino]-benzoic Acid Methyl Ester

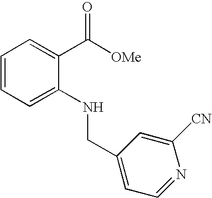

1.28 g (4.0 mmol) of 2-[(2-bromo-pyridin-4-ylmethyl)-amino]-benzoic acid methyl ester in 140 ml of dimethylacetamide is mixed with 0.532 g (4.56 mmol) of zinc(II) cyanide, 0.072 g (0.08 mmol) of tris-(dibenzylideneacetone)-dipalladium, 0.088 g (0.16 mmol) of bis-(diphenylphosphino)-ferrocene and 0.029 g (0.46 mmol) of zinc powder. It is stirred for 6 hours at 150° C. After cooling, the reaction mixture is poured into water. It is extracted three times with ethyl acetate; the combined organic phases are dried on sodium sulfate and concentrated by evaporation. The reaction product is chromatographed on silica gel with a gradient that consists of hexane:ethyl acetate 100:0 to 50:50 as an eluant. 0.887 g (83% of theory) of 2-[(2-cyano-pyridin-4-ylmethyl)-amino]-benzoic acid methyl ester is obtained in the form of a yellow solid.

3. Process Stage

F-3) 2-[(2-Cyano-pyridin-4-ylmethyl)amino]-N-(7-methoxy-3-methyl-quinolin-2-yl)-benzamide

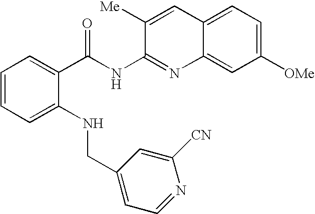

At 0° C., 0.25 ml of trimethylaluminum (2 M in toluene) is added in drops to 0.094 g (0.5 mmol) of 7-methoxy-3-methyl-quinolin-2-ylamine in 4 ml of toluene. After 10 minutes of continuous stirring at 0° C., 0.133 g (0.5 mmol) of 2-[(2-cyano-pyridin-4-ylmethyl)-amino]-benzoic acid methyl ester in 2 ml of toluene is added in drops. Then, it is refluxed for 2 hours and stirred overnight at room temperature. The precipitate is suctioned off and suspended in saturated sodium bicarbonate solution. Then, ethylenediaminetetraacetic acid is added. It is shaken out with ethyl acetate, dried on sodium sulfate and concentrated by evaporation. Column-chromatographic purification on silica gel with a gradient of hexane:acetone=100:0 to 50:50 as an eluant yields 0.113 g (54% of theory) of 2-[(2-cyano-pyridin-4-ylmethyl)-amino]-N-(7-methoxy-3-methyl-quinolin-2-yl)-benzamide as a yellow foam.

### EXAMPLE G

1. Process Stage G1) Production of 2-[(2-Bromo-pyridin-4-ylmethyl)-amino]-benzoic Acid

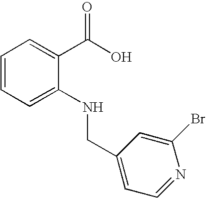

10.0 g (31.2 mmol) of 2-[(2-bromo-pyridin-4-ylmethyl)-amino]-benzoic acid methyl ester is dissolved in 290 ml of ethanol and mixed with 31.2 ml of 2 M sodium hydroxide solution. After it has been stirred overnight at room temperature, the ethanol is drawn off, and the aqueous phase is shaken out with ethyl acetate. The aqueous phase is acidified with concentrated hydrochloric acid. The precipitate that is formed is suctioned off and dried. 5.93 g (62%) of 2-[(2-bromo-pyridin-4-ylmethyl)-amino]-benzoic acid accumulates in the form of a white solid.

2. Process Stage

G2) Production of 2-[(2-Bromo-pyridin-4-ylmethyl)-amino]-N-(2-methyl-2H-indazol-6-yl)-benzamide

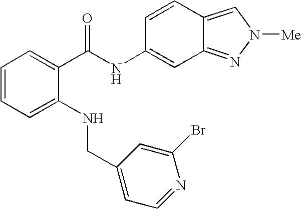

0.500 g (1.6 mmol) of 2-[(2-bromo-pyridin-4-ylmethyl)-amino]-benzoic acid, 0.471 g (3.2 mmol) of 2-methyl-2H-indazol-6-ylamine, 0.4 ml (3.68 mmol) of N-methylmorpholine and 0.729 g (1.92 mmol) of O-(7-azabenzotriazol-1-yl)-1,1,3,3-tetramethyluroniumhexafluorophosphate (HATU) in 25 ml of dimethylformamide are stirred for 16 hours at room temperature. The dimethylformamide is drawn off in an oil pump vacuum. The remaining residue is taken up in saturated sodium bicarbonate solution. It is extracted three times with ethyl acetate, and the combined organic phases are dried, filtered and concentrated by evaporation. The residue is chromatographed on silica gel with a gradient that consists of hexane:acetone=100:0 to 50:50 as an eluant. 0.669 g (96% of theory) of 2-[(2-bromo-pyridin-4-ylmethyl)-amino]-N-(2-methyl-2H-indazol-6-yl)-benzamide is obtained in the form of a beige foam.

Similarly produced are:

G3) 2-[(2-Bromo-pyridin-4-ylmethyl)-amino]-N-(1-methyl-1H-indazol-6-yl)-benzamide

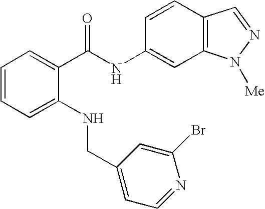

G4) 2-[(2-Bromo-pyridin-4-ylmethyl)-amino]-N-(1H-indazol-6-yl)-benzamide

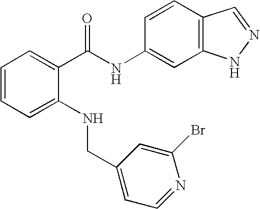

G-5) 2-[(2-Bromo-pyridin-4-ylmethyl)-amino]-N-(1H-indazol-5-yl)-benzamide

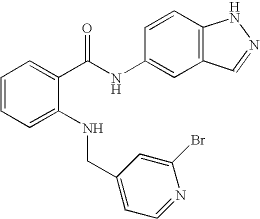

G6) 2-[(2-Bromo-pyridin-4-ylmethyl)-amino]-N-(2-oxo-2,3-dihydro-1H-indol-6-yl)-benzamide

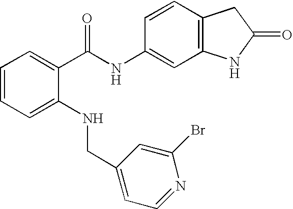

G-7) 2-[(2-Bromo-pyridin-4-ylmethyl)-amino]-N-(2-oxo-2,3-dihydro-1H-indol-5-yl)-benzamide

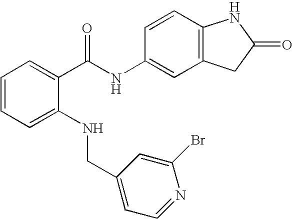

H-1) 4-(tert-Butoxycarbonylamino-methyl)-pyridine-2-carboxylic Acid

4-(tert-Butoxycarbonylamino-methyl)-pyridine-2-carboxylic acid is produced according to Chem. Eur. J. 2, 2000, 216 from (2-cyano-pyridin-4-ylmethyl)-carbamic acid-tert-butyl ester.

H-2) Optically Active [2-(2-Hydroxy-propylcarbamoyl)-pyridin-4-ylmethyl]-carbamic Acid-tert-butyl Ester

Optically active [2-(2-hydroxy-propylcarbamoyl)-pyridin-4-ylmethyl]-carbamic acid-tert-butyl ester is produced according to the process, provided in Example 2.0, from 4-(tert-butoxycarbonylamino-methyl)-pyridine-2-carboxylic acid and S-(+)-1-amino-2-propanol in a yield of 91%.

H-3) Optically Active 4-Aminomethyl-pyridine-2-carboxylic Acid (2-Hydroxy-propyl)-amide

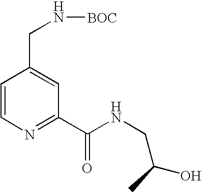

480 mg (1.7 mmol) of [2-(2-hydroxy-propylcarbamoyl)-pyridin-4-ylmethyl]-carbamic acid-tert-butyl ester is mixed in 30 ml of ethanol with 17 ml of 1N hydrochloric acid and heated to a bath temperature of 110° C. for three hours while nitrogen is passing through it. The batch is concentrated by evaporation in a vacuum and dried and used in Example 5.0 without further purification.

H-4) 2-Chloro-N-isoquinolin-3-yl-nicotinamide

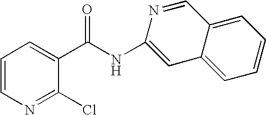

2.9 g (20 mmol) of 3-aminoisoquinoline is suspended in 45 ml of tetrahydrofuran and mixed drop by drop with a solution of 3.5 g (20 mmol) of 2-chloro-nicotinoyl chloride in 45 ml of tetrahydrofuran. After stirring overnight at room temperature, the batch is suctioned off, and the residue is rewashed with tetrahydrofuran. The residue is suspended and suctioned off again as well as dried. 3.14 g (55% of theory) of 2-chloro-N-isoquinolin-3-yl-nicotinamide is obtained.

The sample applications below explain the biological action and the use of the compounds according to the invention without the latter being limited to the examples.

Solutions Required for the Tests

Stock solutions

Stock solution A: 3 mmol of ATP in water, pH 7.0 (−70° C.)

Stock solution B: g-33 P-ATP 1 mCi/100 μl

Stock solution C: poly-(Glu4 Tyr) 10 mg/ml in water

Solution for dilutions

Substrate solvent: 10 mmol of DTT, 10 mmol of manganese chloride, 100 mmol of magnesium chloride

Enzyme solution: 120 mmol of tris/HCl, pH 7.5, 10 μM of sodium vanadium oxide

Sample Application 1

Inhibition of the KDR- and FLT-1 Kinase Activity in the Presence of the Compounds According to the Invention

In a microtiter plate (without protein binding) that tapers to a point, 10 μl of substrate mix (10 μl of volume of ATP stock solution A+25 μCi of g-33P-ATP (about 2.5 μl of stock solution B)+30 μl of poly-(Glu4Tyr) stock solution C+1.21 ml of substrate solvent), 10 μl of inhibitor solution (substances corresponding to the dilutions, 3% DMSO in substrate solvent as a control) and 10 μl of enzyme solution (11.25 μg of enzyme stock solution (KDR or FLT-1 kinase) are added at 4° C. in 1.25 ml of enzyme solution (dilute). It is thoroughly mixed and incubated for 10 minutes at room temperature. Then, 10 μl of stop solution (250 mmol of EDTA, pH 7.0) is added, mixed, and 10 μl of the solution is transferred to a P 81 phosphocellulose filter. Then, it is washed several times in 0.1 M phosphoric acid. The filter paper is dried, coated with Meltilex and measured in a microbeta counter.

The IC50 values are determined from the inhibitor concentration, which is necessary to inhibit the phosphate incorporation to 50% of the uninhibited incorporation after removal of the blank reading (EDTA-stopped reaction).

The results of the kinase inhibition IC50 in nM are presented in the table below:

| | VEGFR II (KDR) |
|---|---|
| Example No. | [nM] |
| 1.32 | 40 |

Sample Application 2

Cytochrome P450 Inhibition

The Cytochrome P450 inhibition was performed according to the publication of Crespi et al. (Anal. Biochem., 248, 188-190 (1997)) with use of baculovirus/insect cell-expressed, human Cytochrome P 450 isoenzymes (1A2, 2C9, 2C19, 3A4).

The results are presented in the following table.

Inhibition of the Cytochrome P450 Isoenzymes (IC50, μM)

| Cytochrome | 1A2 | 2C9 | 2C19 | 3A4 |
|---|---|---|---|---|
| P450 | | | | |
| Isoenzyme | | | | |
| Example | 5.2 | 0.2 | 0.05 | 3.6 |
| 2.54 of WO | | | | |
| 00/27819 | | | | |
| Example | 30 | 2.9 | 4.9 | 25 |
| 1.32 | | | | |

The superior action of the compounds according to the invention compared to the known compounds can be seen clearly from the result, i.e., the compounds according to the invention show a significantly slighter inhibition of the detoxifying P450 system than the known compounds, which results in significantly fewer interactions with other active ingredients.

1. Compounds of general formula I 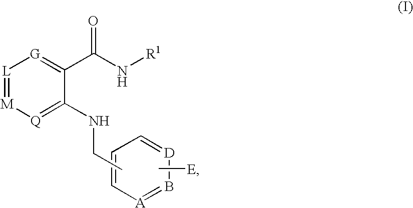
in which
A, B and D, independently of one another, stand for a nitrogen or carbon atom, whereby at least one nitrogen atom is contained in the ring,
E stands for aryl or hetaryl that is optionally substituted in one or more places in the same way or differently with halogen, cyano, C₁-C₆-alkyl, C₁-C₆-alkoxy, halo-C₁-C₆-alkyl or with the group —OR⁵, —SR⁴, —SOR⁴ or —SO₂R⁴, or for the group —COOR⁸, —CONR²R³, —SR⁴, —SOR⁴, —SO₂R⁴, —SCN, —PO(OR¹²)(OR¹³), —CH═CH—COR⁹ or —C≡C—R⁹,
G stands for a nitrogen atom or for the group —C—X,
L stands for a nitrogen atom or for the group —C—X,
M stands for a nitrogen atom or for the group —C—X,
Q stands for a nitrogen atom or for the group —C—X, whereby at most one nitrogen atom is in the ring,
X stands for hydrogen, halogen or for C₁-C₆-alkyl, C₁-C₆-alkyloxy or C₁-C₆-carboxyalkyl that is unsubstituted or optionally substituted in one or more places with halogen,
R¹ stands for branched or unbranched C₁-C₁₂-alkyl or C₂-C₁₂-alkenyl that is optionally substituted in one or more places in the same way or differently with halogen, hydroxy, C₁-C₆-alkyloxy, aralkyloxy, C₁-C₆-alkyl and/or with the group —NR²R³; or for C₃-C₁₀-cycloalkyl or C₃-C₁₀-cycloalkenyl that is optionally substituted in one or more places in the same way or differently with halogen, hydroxy, C₁-C₆-alkyloxy, C₁-C₆-alkyl and/or with the group —NR²R³; or for aryl or hetaryl that is optionally substituted in one or more places in the same way or differently with halogen, cyano, hydroxy, C₁-C₆-alkyloxy, C₂-C₆-alkenyl, aryl-C₁-C₆-alkyloxy, aralkyloxy, C₁-C₆-alkyl, halo-C₁-C₆-alkyl or with the group ═O, —SO₂R⁴, OR⁵, —R⁵ or —PO(OR¹²)(OR¹³),
R² and R³, independently of one another, stand for hydrogen or for C₁-C₆-alkyl, C₃-C₆-cycloalkyl, C₃-C₆-cycloalkenyl, aryl or hetaryl that is optionally substituted in one or more places in the same way or differently with halogen, cyano, C₁-C₆-alkyl, phenyl, hydroxy-C₁-C₆-alkyl, halo-C₁-C₆-alkyl, or with the group —NR⁶R⁷, —OR⁵, C—C₆-alkyl-OR⁵, —SR⁴, —SOR⁴ or —SO₂R⁴, or
R² and R³, together with the nitrogen atom, form a C₃-C₈-ring, which optionally can contain another nitrogen, sulfur or oxygen atom in the ring, or can contain the group —N(R¹⁰), and which optionally can be substituted in one or more places in the same way or differently with halogen, cyano, C₁-C₆-alkyl, halo-C₁-C₆-alkyl, aryl or with the group —OR 5-SR⁴, —SOR⁴ or —SO₂R⁴,
R⁴ stands for hydroxy, C₁-C₆-alkyl, aryl, heteroaryl or for the group —NR²R³,
R⁵ stands for hydrogen, C₁-C₁₂-alkyl, halo-C₁-C₆-alkyl, C₃-C₆-cycloalkyl or halo-C₃-C₆-cycloalkyl, or for C₁-C₁₂-alkyl, which is interrupted in one or more places with oxygen or stands for the group —(CH₂)₂NR²R³, —CH₂CN or —CH₂CF₃,
R⁶ and R⁷, independently of one another, stand for hydrogen or C₁-C₆-alkyl, or
R⁶ and R⁷ together form a 5- to 7-membered ring that can contain an oxygen or sulfur atom or the group —N(R¹⁰)—,
R⁸ stands for hydrogen or for C₁-C₆-alkyl, C₁-C₆-alkoxy, benzyl, aryl or hetaryl that is optionally substituted with halogen in one or more places,
R⁹ stands for hydrogen, C₁-C₆-alkyl, tri-C₁₋₆-alkylsilyl, aryl, hetaryl, or for the group —COR¹¹,
R¹⁰ stands for hydrogen, C₁-C₆-alkyl or aryl,
R¹¹ stands for hydrogen, C₁-C₆-alkyl or for the group —NR²R³, and
R¹² and R³, independently of one another, stand for hydrogen or C—C₆-alkyl, as well as isomers, enantiomers and salts thereof.

2. Compounds of general formula I, according to claim 1, in which
A, B, and D, independently of one another, stand for a nitrogen or carbon atom, whereby at least one nitrogen atom is contained in the ring,
E stands for aryl or hetaryl that is optionally substituted in one or more places in the same way or differently with halogen, cyano, C₁-C₆-alkyl, C₁-C₆-alkoxy, halo-C₁-C₆-alkyl or with the group —OR⁵, —SR⁴, —SOR⁴ or —SO₂R⁴, or for the group —COOR⁸, —CONR²R³, —SR⁴, —SOR⁴, —SO₂R⁴, —SCN, —PO(OR¹²)(OR¹³), —CH═CH—COR⁹ or —C≡C—R⁹,
G stands for a nitrogen atom or for the group —C—X,
L stands for a nitrogen atom or for the group —C—X,
M stands for a nitrogen atom or for the group —C—X,
Q stands for a nitrogen atom or for the group —C—X,
whereby at most one nitrogen atom is in the ring,
X stands for hydrogen, halogen or for C₁-C₆-alkyl, C₁-C₆-alkyloxy or C₁-C₆-carboxyalkyl that is unsubstituted or that is optionally substituted in one or more places with halogen,
R¹ stands for aryl or hetaryl that is optionally substituted in one or more places in the same way or differently with halogen, cyano, hydroxy, C₁-C₆-alkyloxy, C₂-C₆-alkenyl, aryl-C₁-C₆-alkyloxy, aralkyloxy, C₁-C₆-alkyl, halo-C₁-C₆-alkyl or with the group ═O, —SO₂R⁴, OR⁵, —R⁵ or —PO(OR¹²)(OR¹³),
R² and R³, independently of one another, stand for hydrogen or for C₁-C₆-alkyl, C₃-C₆-cycloalkyl, C₃-C₆-cycloalkenyl, aryl or hetaryl that is optionally substituted in one or more places in the same way or differently with halogen, cyano, C₁-C₆-alkyl, phenyl, hydroxy-C₁-C₆-alkyl, halo-C₁-C₆-alkyl or with the group —NR⁶R⁷, —OR⁵, C₁-C₆-alkyl-OR⁵, —SR⁴, —SOR⁴ or —SO₂R⁴, or
R² and R³ together with the nitrogen atom form a C₃-C₈ ring, which optionally can contain another nitrogen, sulfur or oxygen atom in the ring, or can contain the group —N(R¹⁰), and which optionally can be substituted in one or more places in the same way or differently with halogen, cyano, C₁-C₆-alkyl, halo-C₁-C₆-alkyl, aryl or with the group —OR⁵, —SR⁴, —SOR⁴ or
—SO₂R⁴,
R⁴ stands for hydroxy, C₁-C₆-alkyl, aryl, heteroaryl or for the group —NR²R³,
R⁵ stands for hydrogen, C₁-C₁₂-alkyl, halo-C₁-C₆-alkyl, C₃-C₆-cycloalkyl or halo-C₃-C₆-cycloalkyl, or for C₁-C₁₂-alkyl, which is interrupted in one or more places with oxygen, or stands for the group —CH₂)₂NR²R³, —CH₂CN or —CH₂CF₃,
R⁶ and R⁷, independently of one another, stand for hydrogen or C₁-C₆-alkyl,
or R⁶ and R⁷ together form a 5- to 7-membered ring, which can contain an oxygen or sulfur atom or the group —N(R¹⁰)—,
R⁸ stands for hydrogen or for C₁-C₆-alkyl, C₁-C₆-alkoxy, benzyl, aryl or hetaryl that is optionally substituted with halogen in one or more places,
R⁹ stands for hydrogen, C₁-C₆-alkyl, tri-C₁-C₆-alkylsilyl, aryl, hetaryl or for the group —COR¹¹,
R¹⁰ stands for hydrogen, C₁-C₆-alkyl or aryl,
R¹¹ stands for hydrogen, C₁-C₆-alkyl or for the group —NR²R³, and
R¹² and R³, independently of one another, stand for hydrogen or C—C₆-alkyl, as well as isomers, enantiomers and salts thereof.

3. Compounds of general formula I, according to claim 1 and 2, in which
A, B and D, independently of one another, stand for a nitrogen or carbon atom, whereby at least one nitrogen atom is contained in the ring,
E stands for aryl or hetaryl that is optionally substituted in one or more places in the same way or differently with halogen, cyano, C₁-C₆alkyl, C₁-C₆alkoxy, halo-C₁-C₆alkyl or with the group —OR⁵, —SR⁴, —SOR⁴ or —SO₂R⁴, or for the group —COOR⁸, —CONR²R³, —SR⁴, —SOR⁴, —SO₂R⁴, —SCN, —PO(OR¹²)(OR¹³), —CH═CH—COR⁹ or —C≡C—R⁹,
G stands for a nitrogen atom or for the group —C—X,
L stands for a nitrogen atom or for the group —C—X,
M stands for a nitrogen atom or for the group —C—X,
Q stands for a nitrogen atom or for the group —C—X, whereby at most one nitrogen atom is in the ring,
X stands for hydrogen or halogen,
R¹ stands for aryl or hetaryl that is optionally substituted in one or more places in the same way or differently with halogen, hydroxy, C₁-C₆-alkyloxy, aralkyloxy, C₁-C₆-alkyl, halo-C₁-C₆-alkyl or with the group —SO₂R⁴, OR⁵, —R⁵ or —PO(OR¹²)(OR¹³),
R² and R³, independently of one another, stand for hydrogen or for C₁-C₆-alkyl, C₃-C₆-cycloalkyl, C₃-C₆-cycloalkenyl, aryl or hetaryl that is optionally substituted in one or more places in the same way or differently with halogen, cyano, C₁-C₆-alkyl, phenyl, hydroxy-C₁-C₆-alkyl, halo-C₁-C₆-alkyl or with the group —NR⁶R⁷—OR⁵, C₁-C₆-alkyl-OR⁵, —SR⁴, —SOR⁴ or —SO₂R⁴, or
R² and R³ together with the nitrogen atom form a C₃-C₈-ring, which optionally can contain another nitrogen, sulfur or oxygen atom in the ring, or can contain the group —N(R¹⁰), and which optionally can be substituted in one or more places in the same way or differently with halogen, cyano, C₁-C₆-alkyl, halo-C₁-C₆-alkyl, aryl or with the group —OR⁵, —SR⁴, —SOR⁴ or —SO₂R⁴,
R⁴ stands for hydroxy or for the group —NR²R³,
R⁵ stands for hydrogen, C₁-C₁₂-alkyl or for C₁-C₁₂-alkyl, which is interrupted in one or more places with oxygen or stands for the group —(CH₂)₂NR²R³, —CH₂CN or —CH₂CF₃,
R⁶ and R⁷, independently of one another, stand for hydrogen or C₁-C₆-alkyl, or
R⁶ and R⁷ together form a 5- to 7-membered ring, which can contain an oxygen or sulfur atom or the group —N(R¹⁰)—,
R⁸ stands for hydrogen or for C₁-C₆-alkyl, C₁-C₆-alkoxy, benzyl, aryl or hetaryl that is optionally substituted with halogen in one or more places,
R⁹ stands for hydrogen, C₁-C₆-alkyl, tri-C₁-C₆-alkylsilyl, aryl, hetaryl or for the group —COR¹¹,
R¹⁰ stands for hydrogen, C₁-C₆-alkyl or aryl,
R¹¹ stands for hydrogen, C₁-C₆-alkyl or for the group —NR²R³, and
R¹² and R³, independently of one another, stand for hydrogen or C₁-C₆-alkyl, as well as isomers, enantiomers and salts thereof.

4. Compounds of general formula I, according to claims 1 to 3 claim 1, in which
A, B and D stand for a nitrogen or carbon atom, whereby at least one nitrogen atom is contained in the ring,
E stands for hetaryl that is optionally substituted in one or more places in the same way or differently with halogen, cyano, C₁₋₆alkyl, C₁-C₆-alkoxy, halo-C₁-C₆-alkyl or with the group —OR⁵, —SR⁴, —SOR⁴ or —SO₂R⁴, or for the group —COOR⁸, —CONR²R³, —SR⁴, —SOR⁴, —SO₂R⁴, —SCN, —PO(OR¹²)(OR¹³), —CH═CH—COR⁹ or —C≡C—R⁹,
G stands for the group —C—X,
L stands for the group —C—X,
M stands for the group —C—X,
Q stands for a nitrogen atom or for the group —C—X,
X stands for hydrogen or halogen,
R¹ stands for phenyl, thiophene, furan, oxazole, thiazole, imidazole, pyrazole, pyridine, pyrimidine, triazine, quinoline, or isoquinoline that is optionally substituted in one or more places in the same way or differently with halogen, hydroxy, C₁-C₆-alkyloxy, aralkyloxy, C₁-C₆-alkyl, halo-C₁-C₆-alkyl or with the group —SO₂R⁴, OR⁵, —R⁵ or —PO(OR¹²)(OR¹³) or is substituted on the group 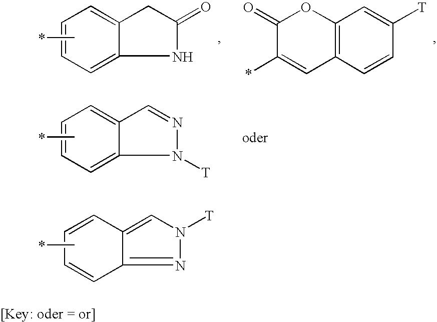
in which T stands for hydrogen, C₁-C₆-alkyl or C₁-C₆-alkoxy,
R² and R³, independently of one another, stand for hydrogen or for C₁-C₆-alkyl, C₃-C₆-cycloalkyl, C₃-C₆-cycloalkenyl, aryl or hetaryl that is optionally substituted in one or more places in the same way or differently with halogen, cyano, C₁-C₆-alkyl, phenyl, hydroxy-C₁-C₆-alkyl, halo-C₁-C₆-alkyl or with the group —NR⁶R⁷, —OR⁵, C₁-C₆-alkyl-OR⁵, —SR⁴, —SOR⁴ or —SO₂R⁴, or
R² and R³, together with the nitrogen atom, form a C₃-C₈-ring, which optionally can contain another nitrogen, sulfur or oxygen atom in the ring, or can contain the group —N(R¹⁰), and which optionally can be substituted in one or more places in the same way or differently with halogen, cyano, C₁-C₆-alkyl, halo-C₁-C₆-alkyl, aryl or with the group —OR⁵, —SR⁴, —SOR⁴ or —SO₂R⁴,
R⁴ stands for hydroxy or for the group —NR²R³,
R⁵ stands for hydrogen, C₁-C₁₂-alkyl or for C₁-C₁₂-alkyl, which is interrupted in one or more places with oxygen, or stands for the group —CH₂)₂NR²R³, —CH₂CN, or —CH₂CF₃,
R⁶ and R⁷, independently of one another, stand for hydrogen or C₁-C₆-alkyl, or
R⁶ and R⁷ together form a 5- to 7-membered ring that can contain an oxygen or sulfur atom,
R⁸ stands for hydrogen or for C₁-C₆-alkyl, C₁-C₆-alkoxy, benzyl, aryl or hetaryl that is optionally substituted in one or more places with halogen, and
R⁹ stands for hydrogen, C₁-C₆-alkyl or tri-C₁-C₆-alkylsilyl, and
R¹² and R³, independently of one another, stand for hydrogen or C₁-C₆-alkyl, as well as isomers, enantiomers and salts thereof.

5. Compounds of general formula I, according to claim 1, in which
A, B and D, independently of one another, stand for a nitrogen or carbon atom, whereby at least one nitrogen atom is contained in the ring,
E stands for thienyl, pyridyl or for the group —COOR⁸, —CONR²R³, or —C≡C—R⁹,
G stands for the group —C—X,
L stands for the group —C—X,
M stands for the group —C—X,
Q stands for a nitrogen atom or for the group —C—X,
X stands for hydrogen or halogen,
R¹ stands for phenyl, thiophene, furan, oxazole, thiazole, imidazole, pyrazole, pyridine, pyrimidine, triazine, quinoline or isoquinoline that is optionally substituted in one or more places in the same way or differently with halogen, hydroxy, C₁-C₆-alkyloxy, aralkyloxy, C₁-C₆-alkyl, halo-C₁-C₆-alkyl or with the group —SO₂R⁴, OR⁵, —R⁵ or —PO(OR¹²)(OR¹³) or substituted on the group 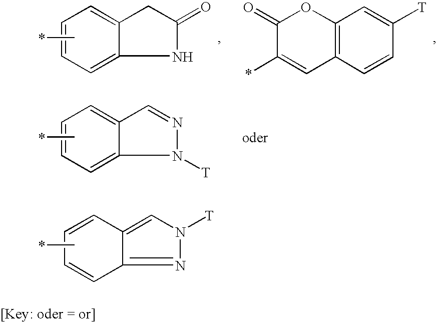
in which T stands for hydrogen, C₁-C₆-alkyl or C₁-C₆-alkoxy,
R² and R³, independently of one another, stand for hydrogen or for C₁-C₆-alkyl, C₃-C₆-cycloalkyl, phenyl or pyridyl that is optionally substituted in one or more places in the same way or differently with halogen, C₁-C₆-alkyl, phenyl or with the group —NR⁶R⁷, —OR⁵ or C₁-C₆-alkyl-OR⁵, or
R² and R³ together with the nitrogen atom form a C₃-C₈-ring, which optionally can contain another nitrogen or oxygen atom in the ring, and which optionally can be substituted in one or more places in the same way or differently with C₁-C₆-alkyl,
R⁴ stands for hydroxy or for the group —NR²R³,
R⁵, R⁶ and R⁷, independently of one another, stand for hydrogen or C₁-C₆-alkyl, or
R⁶ and R⁷ together form a 5- to 7-membered ring, which can contain an oxygen or sulfur atom,
R⁸ stands for hydrogen, C₁-C₆-alkyl or benzyl, and
R⁹ stands for hydrogen, C₁-C₆-alkyl or tri-C₁-C₆-alkylsilyl, and
R¹² and R³, independently of one another, stand for hydrogen or C₁-C₆-alkyl, as well as isomers and salts thereof.

6. Pharmaceutical agents that contain at least one compound according to claim 1.

7. Pharmaceutical agents according to claim 6 for use in the case of psoriasis,
Kaposi's sarcoma, restenosis, such as, e.g., stent-induced restenosis, endometriosis, Crohn's disease, Hodgkin's disease, leukemia; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, inhibition of the reocclusion of vessels after balloon catheter treatment, vascular prosthetics or use of mechanical devices to keep vessels open, such as, e.g., stents, and as immunosuppressive agents, and for supporting scar-free healing, in senile keratosis and in contact dermatitis.

8. Pharmaceutical agents according to claim 6 for use as VEGFR kinase 3-inhibitors
in lymphangiogenesis and in hyper- and dysplastic changes of the lymphatic system.

9. Compounds according to claim 1, with suitable formulation substances and vehicles.

10. Use of the compounds of formula I, according to claim 1, as inhibitors of the tyrosine kinases KDR and FLT.

11. Use of the compounds of general formula I, according to claim 1 in the form of a pharmaceutical preparation for enteral, parenteral and oral administration.

12. Use of the compounds according to claim 1 in the case of
psoriasis, Kaposi's sarcoma, restenosis, such as, e.g., stent-induced restenosis, endometriosis, Crohn's disease, Hodgkin's disease, leukemia; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, and for inhibiting the reocclusion of vessels after balloon catheter treatment, in vascular prosthetics or after mechanical devices are used to keep vessels open, such as, e.g., stents, and as immunosuppressive agents, and for supporting scar-free healing, and in senile keratosis and in contact dermatitis.
